(19) United States
(12) Patent Application Publication
(10) Pub. No.: US 2011/0212099 A1
(43) Pub. Date: Sep. 1, 2011

(54) METHODS OF MODULATING IL-22 AND IL-17

(75) Inventors: Spencer C. LIANG, Mountain View, CA (US); Lynette A. Fouser, Acton, MA (US); Margot O'Toole, Newtonville, MA (US)

(73) Assignee: Wyeth LLC, Madison, NJ (US)

(21) Appl. No.: 13/048,105

(22) Filed: Mar. 15, 2011

Related U.S. Application Data
(63) Continuation of application No. 11/812,310, filed on Jun. 18, 2007, now abandoned.
(60) Provisional application No. 60/814,573, filed on Jun. 19, 2006.

(51) Int. Cl. A61K 39/395; G01N 33/68; C12N 5/071; A61K 38/02; A61P 29/00; A61P 19/02; A61P 17/06; A61P 37/06; A61P 25/28; A61P 1/18; A61P 1/00

(52) U.S. Cl. 424/145.1; 435/7.21; 435/375; 514/1.1; 435/7.1

(57) ABSTRACT

The present application provides methods of modulating immune responses by using IL-22 in combination with at least one of IL-17A, IL-17F, or IL-23 or by using an IL-22 antagonist, such as an antibody or a soluble receptor or a binding protein, in combination with an antagonist of at least one of IL-17A, IL-17F, or IL-23

### CROSS REFERENCE TO RELATED APPLICATIONS

The present application claims the benefit of U.S. provisional application No. 60/814,573, filed Jun. 19, 2006, the entire disclosure of which is relied upon and incorporated by reference.

### TECHNICAL FIELD

This invention relates to methods of modulating immune responses by using IL-22 in combination with at least one of IL-17A, IL-17F, or IL-23 or by using an IL-22 antagonist, such as an antibody or a soluble receptor or a binding protein, in combination with an antagonist of at least one of IL-17A, IL-17F, or IL-23.

### BACKGROUND

The role of CD4 T cells in regulating immune responses and disease is well established. Interleukin-22 (IL-22) is a class II cytokine that is up-regulated in T cells by IL-9 or ConA (Dumoutier et al., Proc. Natl. Acad. Sci. USA (2000) 97 (18):10144-49). One function of IL-22 is to enhance the innate immunity of peripheral tissues by inducing the expression of anti-microbial peptides including beta-defensin 2 (hBD-2), S100A7, S100A8, and S100A9 (Wolk et al., Immunity (2004) 21:241-54; Boniface et al., J. Immunol. (2005) 174:3695-3702). Other studies have shown that expression of IL-22 mRNA is induced in vivo in response to LPS administration, and that IL-22 modulates parameters indicative of an acute phase response (Dumoutier L. et al. (2000); Pittman et al., Genes and Immunity, (2001) 2:172). Taken together, these observations indicate that IL-22 plays a role in inflammation (Kotenko S. V., Cytokine & Growth Factor Reviews (2002) 13 (3):223-40). Several T cell disorders, including psoriasis (Wolk et al., Immunity (2004) 21:241-54), rheumatoid arthritis (Ikeuchi H. et al. Arthritis Rheum 52:1037-1046), and inflammatory bowel disease (Andoh, A. et al. Gastroenterology 129:969-984) are associated with increased levels of IL-22.

Recent data have demonstrated the existence of a new CD4⁺ effector lineage that is defined by its ability to express IL-17A and IL-17F (hereafter referred to as the Th17 lineage) (Aggarwal et al., J. Biol. Chem., (2003) 278:1910-14; Langrish et al., J. Exp. Med., (2005) 201:233-40; Harrington et al., Nat. Immunol., (2005) 6:1123-32; Park et al., Nat. Immunol., (2005) 6:1133-41; Veldhoen et al., Immunity, (2006) 24:179-89; Mangan et al., Nature, (2006) 441:231-34; Bettelli et al., Nature, (2006) 441:235-38). Th17 cell differentiation is initiated by TGF-β signaling in the context of pro-inflammatory cytokines, particularly IL-6, and also IL-1β and TNF-α. Maintenance and survival of Th17 cells, in contrast, are dependent upon IL-23, an IL-12 family member composed of IL-12p40 and IL-23p19 subunits. IL-23 deficient mice produce significantly less IL-17 in several murine disease and infection models (Langrish et al., J. Exp. Med., (2005) 201:233-40; Murphy et al., J. Exp. Med., (2003) 198:1951-57; Happel et al., J. Exp. Med., (2005) 202:761-69; Khader et al., J. Immunol., (2005) 175:788-95). Thus, Th17 differentiation is initiated by TGF-β and pro-inflammatory cytokines and subsequently maintained by IL-23.

The IL-17 family is composed of five family members—IL-17A, IL-17B, IL-17C, IL-17D, IL-17E (IL-25), and IL-17F—that share a relative homology between 17 to 55% (Aggarwal et al., Cytokine Growth Factor Rev., (2003) 14:155-74; Kolls et al., Immunity, (2004) 21:467-76). The expression of IL-17 family members is quite diverse. IL-17A and IL-17F are the most homologous (55%) and are located adjacent to each other on human chromosome 1. IL-17A and IL-17F mRNA are expressed at higher levels in Th17 cells as compared to Th1 or Th2 cells. In contrast, IL-17B, IL-17C, and IL-17D are expressed predominantly in non-lymphoid tissues. IL-17E (IL-25) is expressed in Th2 cells (Fort et al., Immunity, (2001) 15:985-95). In addition to IL-17A and IL-17F, TNF-α, IL-6, and GM-CSF have also been identified as genes induced by IL-23 and potentially expressed by Th17 cells (Langrish et al., J. Exp. Med., (2005) 201:233-40; Infante-Duarte et al., J. Immunol., (2000) 165:6107-15). However, because Th1 cells can express TNF-α and Th2 cells can express IL-6 and GM-CSF, the expression of IL-6, TNF-α, and GM-CSF is not restricted to the Th17 lineage. In contrast, Th17 cells are thought to produce IL-17A and IL-17F in a lineage specific manner.

Subsets of CD4 effector cells are involved in a number of different diseases. In some cases, their activity is helpful to the organism. In other diseases, however, their activity is undesirable or even harmful. Identification of those subsets of cells within the CD4 effector population that are responsible for a particular pathology permits targeted regulation of those cells without unneeded suppression of other CD4 effector cells. Similarly, knowledge of the cytokines produced by cellular subsets and how those cytokines interact is a prerequisite for the development of comprehensive therapies that provide improved management of diseases involving those cytokines. A need therefore exists in the an for further characterization of the cytokines produced by the Th17 lineage of CD4 effector cells.

The present application meets this need by showing that IL-22, an IL-10 family member originally described as a Th1 cytokine, is also a Th17 cytokine that can act cooperatively, and in some cases, synergistically, with IL-17A or IL-17F. In addition, IL-22 induction by IL-23 is demonstrated.

### SUMMARY

The present application provides methods of modulating immune responses by using interleukin-22 (“IL-22”) in combination with at least one of interleukin-17A (“IL-17A”), interleukin-17F (“IL-17F”), or interleukin-23 (“IL-23”) or by using an IL-22 antagonist, such as an antibody or a soluble receptor or a binding protein, in combination with an antagonist of at least one of IL-17A, IL-17F, or IL-23.

In one embodiment, the methods comprise diagnosing, preventing, and/or treating diseases associated with IL-22 and least one of IL-17A, IL-17F, or IL-23. This can be accomplished, at least in part, through the use of compositions comprising two or more antagonists, such as antibodies, soluble receptors, or binding proteins, that inhibit IL-22 and at least one of IL-17A, IL-17F, or IL-23.

The compositions and combinations of antagonists used for preventing and/or treating diseases decrease the activity of IL-22 and at least one of IL-17A, IL-17F, or IL-23. For example, the activity of any cytokine can be reduced or inhibited by contacting it with a composition comprising an antibody that binds to the cytokine and inhibits its function. The functional activity of a cytokine can also be affected by reducing or inhibiting its signaling through cellular receptors using agents, such as antibodies or soluble receptors, that inhibit or reduce signaling through a cytokine receptor.

The application also provides methods of stimulating an immune response by administering IL-22 and at least one of IL-17A, IL-17F, or IL-23. Stimulation of an immune response may be desirable, for example, when a mammal is infected by a pathogen, such as a bacterium or virus, or when immunogens are administered to a mammal as part of a vaccine. Thus, in one embodiment, the application provides a method of inducing the expression of an anti-microbial peptide in a cell, such as a keratinocyte, comprising administering IL-22 and IL-17A, IL-22 and IL-17F, IL-22 and IL-23, or IL-22, IL-17A, and IL-17F to the cell. The anti-microbial peptide can be, for example, a member of the beta-defensin family, including human beta-defensin 1 or human beta-defensin 2, a member of the S100 family of calcium binding proteins, including S100A7, S100A8, or S100A9, a cathelicidin, including human cathelicidin LL-37 (see Lee et al., PNAS (2005) 102:3750-55), or a combination thereof. Other embodiments are directed to methods of inducing an anti-microbial peptide, comprising administering to a mammal, such as a human, IL-22 and IL-17A, IL-22 and IL-17F, or IL-22, IL-17A, and IL-17F in amounts effective to induce the anti-microbial peptide in the mammal. Still other embodiments are directed to methods of inhibiting or reducing the expression of an anti-microbial peptide in a cell, such as a keratinocyte, comprising administering an antagonist of IL-22, or an antagonist of IL-22 and an antagonist of IL-17A, an antagonist of IL-22 and an antagonist IL-17F, or an antagonist of IL-22, an antagonist of IL-17A, and an antagonist of IL-17F to the cell. Another embodiment is directed to a method of inhibiting or reducing the expression of an anti-microbial peptide, comprising administering to a mammal, such as a human, an antagonist of IL-22, or an antagonist of IL-22 and an antagonist of IL-17A, an antagonist of IL-22 and an antagonist IL-17F, or an antagonist of IL-22, an antagonist of IL-17A, and an antagonist of IL-17F in amounts effective to inhibit or reduce the expression of the anti-microbial peptide. In another embodiment, IL-22 and at least one of IL-17A, IL-17F, or IL-23, are used as an adjuvant. For example, the adjuvants can comprise IL-22 and IL-17A, IL-22 and IL-17F, IL-22 and IL-23, or IL-22, IL-17A, and IL-17F. Immunogens of interest in a vaccine can be, for example, viral, bacterial, or tumor antigens. This application also provides kits comprising the adjuvants discussed herein, either alone, or combined with an immunogen.

Compositions used for diagnosing diseases associated with IL-22 and at least one of IL-17A, IL-17F, or IL-23 need only detect the cytokine proteins or nucleic acids expressing the cytokines. Antibodies and soluble receptors are examples of agents that can be used in compositions to detect cytokine proteins. The nucleic acid expressing a cytokine protein can be detected by a variety of standard techniques, such as polymerase chain reaction (PCR).

In one aspect, the method comprises treating a subject with a disorder associated with IL-22 and at least one of IL-17A, IL-17F, or IL-23. The methods include administering to the subject a composition in an amount sufficient to reduce or inhibit the activity of IL-22 and at least one of IL-17A, IL-17F, or IL-23, thereby treating the disorder. In some embodiments, the composition comprises an IL-22 antagonist, and an antagonist of at least one of IL-17A, IL-17F, or IL-23. In still other embodiments, the composition comprises a combination of one or more antibodies and one or more soluble receptors or binding proteins.

Antagonists that can be used in the invention include antibodies; soluble receptors, including truncated receptors, natural soluble receptors, or fusion proteins comprising a receptor (or a fragment thereof) fused to a second protein, such as an Fc portion of an immunoglobulin; peptide inhibitors; small molecules; ligand fusions; and binding proteins. Examples of binding proteins include the naturally-occurring IL-22 binding proteins (or fragments thereof) described in US2003/0170839, the contents of which are incorporated by reference in its entirety. Small Modular Immunopharmaceutical (SMIP™) (Trubion Pharmaceuticals, Seattle, Wash.) provide an example of a variant molecule comprising a binding domain polypeptide. SMIPs and their uses and applications are disclosed in, e.g., U.S. Published Patent Application. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

A SMIP™ typically refers to a binding domain-immunoglobulin fusion protein that includes a binding domain polypeptide that is fused or otherwise connected to an immunoglobulin hinge or hinge-acting region polypeptide, which in turn is fused or otherwise connected to a region comprising one or more native or engineered constant regions from an immunoglobulin heavy chain, other than CH1, for example, the CH2 and CH3 regions of IgG and IgA, or the CH3 and CH4 regions of IgE (see e.g., U.S. 2005/0136049 by Ledbetter, J. et al., which is incorporated by reference, for a more complete description). The binding domain-immunoglobulin fusion protein can further include a region that includes a native or engineered immunoglobulin heavy chain CH2 constant region polypeptide (or CH3 in the case of a construct derived in whole or in part from IgE) that is fused or otherwise connected to the hinge region polypeptide and a native or engineered immunoglobulin heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-immunoglobulin fusion proteins are capable of at least one immunological activity selected from the group consisting of antibody dependent cell-mediated cytotoxicity, complement fixation, and/or binding to a target, for example, a target antigen, such as human IL-22, IL-17A, IL-17F, or IL-23.

In one embodiment, the antagonist is a VHH molecule (or nanobody), which, as known to the skilled artisan, is a heavy chain variable domain derived from immunoglobulins naturally devoid of light chains, such as those derived from Camelidae as described in WO 9404678 and U.S. Pat. No. 5,759,808, both of which are incorporated herein by reference. Such a VHH molecule can be derived from antibodies raised in Camelidae species, for example in camel, llama, dromedary, alpaca and guanaco and is sometimes called a camelid or camelized variable domain. See e.g., Muyldermans., J. Biotechnology (2001) 74 (4):277-302, incorporated herein by reference. Other species besides Camelidae may produce heavy chain antibodies naturally devoid of light chain. VHH molecules are about 10 times smaller than IgG molecules. They are single polypeptides and very stable, resisting extreme pH and temperature conditions. Moreover, they are resistant to the action of proteases which is not the case for conventional antibodies. Furthermore, in vitro expression of VHHs produces high yield, properly folded functional VHHs. In addition, antibodies generated in Camelids will recognize epitopes other than those recognized by antibodies generated in vitro through the use of antibody libraries or via immunization of mammals other than Camelids (see WO 9749805 and U.S. Patent Application Publication 2004/0248201, both of which are incorporated herein by reference).

Thus, in one embodiment, the composition comprises a first antibody that binds to IL-22 and a second antibody that binds to either IL-17A, IL-17F, or IL-23. In another embodiment, the composition comprises an antibody that binds to IL-22 and a soluble receptor (or binding protein) that binds to IL-17A, IL-17F, or IL-23. In yet another embodiment, the composition comprises a soluble receptor that binds to IL-22 and an antibody or soluble receptor (or binding protein) that bind to IL-17A, IL-17F, or IL-23. In a further embodiment, the composition comprises an IL-22 binding protein and an antibody or soluble receptor (or binding protein) that binds to IL-17A, IL-17F, or IL-23.

The compositions can be administered to the subject, either alone or in combination with additional therapeutic agents as described herein. The subject may be a mammal, e.g. human. In some embodiments, the composition is administered locally, e.g., topically, subcutaneously, or other administrations that are not in the general circulation. In other embodiments, the composition is administered to the general circulation, for example, by intravenous (i.v.) or subcutaneous (s.c.) administration. The different agonists and antagonists may be administered simultaneously or sequentially.

Examples of disorders associated with one or more of IL-22, IL-17A, IL-17F, or IL-23 include respiratory disorders, inflammatory disorders, and autoimmune disorders. In particular, disorders associated with one or more of IL-22, IL-17A, IL-17F, or IL-23 include arthritis (including rheumatoid arthritis, juvenile rheumatoid arthritis, osteoarthritis, psoriatic arthritis, lupus-associated arthritis or ankylosing spondylitis), scleroderma, systemic lupus erythematosis, vasculitis, multiple sclerosis, autoimmune thyroiditis, dermatitis (including atopic dermatitis and eczematous dermatitis), myasthenia gravis, inflammatory bowel disease (IBD), Crohn's disease, colitis, diabetes mellitus (type I); inflammatory conditions of, e.g., the skin (e.g., psoriasis), cardiovascular system (e.g., atherosclerosis), nervous system (e.g., Alzheimer's disease), liver (e.g., hepatitis), kidney (e.g., nephritis) and pancreas (e.g., pancreatitis); cardiovascular disorders, e.g., cholesterol metabolic disorders, oxygen free radical injury, ischemia; disorders associated with wound healing; respiratory disorders, e.g., asthma and COPD (e.g., cystic fibrosis); acute inflammatory conditions (e.g., endotoxemia, septicemia, toxic shock syndrome and infectious disease); transplant rejection and allergy.

In yet another aspect, the application provides methods of treating psoriasis by administering to a psoriasis patient a composition comprising an IL-17F antagonist, such as an antibody or a. soluble receptor in therapeutically effective amounts. The IL-17F antagonist may be administered alone or in combination with an IL-22 antagonist, such as an antibody, soluble receptor, or binding protein.

In another aspect, the application provides a method for detecting the presence of IL-22 and at least one of IL-17A, IL-17F, or IL-23 in a sample in vitro. Samples may include biological materials such as blood, serum, plasma, tissue, biopsy, and bronchoalveolar lavage. The subject method can be used to diagnose a disorder, such as a disorder associated with one or more of IL-22, IL-17A, IL-17F, or IL-23, as described in this application. Such a method can include: (1) contacting the sample or a control sample with a first reagent that binds to IL-22 and a second reagent that binds to IL-17A, IL-17F, or IL-23, and (2) detecting formation of a complex between the first and second reagents and the sample or the control sample, wherein a statistically significant change in the formation of the complex in the sample relative to a control sample, is indicative of the presence of the cytokines in the sample. In one embodiment, the method includes contacting a sample comprising cells with a labeled regeant, such as a fluorescent antibody, that binds to IL-22, IL-17A, IL-17F, or IL-23 within the cells. The amount of reagent detected within a cell is proportional to the amount of intracellular IL-22, IL-17A, IL-17F, or IL-23 expressed within the cell.

In yet another aspect, the application provides an in vivo detection method (e.g., in vivo imaging in a subject). The method can be used to diagnose a disorder, including those disorders described in this application. Such a method can include: (1) administering a first reagent that binds to IL-22 and a second reagent that binds to IL-17A, IL-17F, or IL-23 to a subject or a control subject under conditions that allow binding of the first and second reagents to their cytokines, and (2) detecting formation of a complex between the first and second reagents and their cytokines, wherein a statistically significant change in the formation of the complex in the subject relative to a control, e.g., a control subject, is indicative of the presence of the cytokines.

Examples of reagents that bind to cytokines used in the methods of the invention include antibodies, soluble receptors, and binding proteins. These reagents may be directly or indirectly labeled with a detectable substance to facilitate detection. Suitable detectable substances include various enzymes, prosthetic groups, fluorescent materials, luminescent materials and radioactive materials.

Additional aspects of the disclosure will be set forth in part in the description, and in part will be obvious from the description, or may be learned by practicing the invention. Certain embodiments are set forth and particularly pointed out in the claims, and the disclosure should not be construed as limiting the scope of the claims. The following detailed description includes exemplary representations of various embodiments, which are not restrictive of the subject matter claimed. The accompanying figures constitute a part of this specification and, together with the description, serve only to illustrate embodiments and not limit the invention.

### BRIEF DESCRIPTION OF THE FIGURES

FIG. 1. Cytokine transcript expression profiles for Th1, Th2 and Th17 cells. (A) Quantitative PCR analysis of relative cytokine expression in cells induced to differentiate into Th1, Th2, and Th17 cells. (B) Relative IL-22 levels induced in Th1, Th2, and Th17 cells. (C) Relative levels of IL-2, IL-3, IL-5, IL-6, IL-9, IL-10, IL-13, IL-21, IL-24, IL-25, and IL-31 in Th1, Th2, and Th17 cells. (D) Relative levels of IL-1, IL-7, IL-11, IL-15, IL-16, IL-18, IL-19, IL-20, IL-27, and IL-28 in Th1, Th2, and Th17 cells.

FIG. 2. Expression levels of IL-22 and IL-17A protein in T cell subsets. (A) Levels of IL-22, IL-17A and IFN-γ protein following activation in the presence of various cytokines, antibodies, and antigen. (B) IL-22 levels in differentiated cells restimulated with antigen and various cytokines and antibodies.

FIG. 3. Effects of exogenous IL-22 addition. (A) Levels of IL-22R1 transcripts in the indicated populations following addition of exogneous IL-22. (B) Proliferation of naïve cells in response to exogenous IL-22. (C) IFN-γ production by Th1 cells in response to exogenous IL-22. (D) IL-4 production by Th2 cells in response to exogenous IL-22. (E) IL-17 production by Th17 cells in response to exogenous IL-22.

FIG. 4. Intracellular cytokine levels in T cell populations. (A) Flow cytometric analysis of IL-22 co-expression with IFN-γ or IL-17A in Th1, Th2, and Th17 cells. (B) Flow cytometric analysis of IL-17A and IL-17F co-expression in IL-22-expressing CD4 cells cultured in the presence of the indicated cytokines. (C) Effect of anti-TGF-β addition on IL-22 levels.

FIG. 5. Expansion of IL-22-producing cells by IL-23. (A) Intracellular staining for IL-22 in naïve T cells cultured with antigen and the indicated cytokines. The the graph shows the percentage IL-22 cells in the culture as a function of time while the dot plots show IL-22 and IL-17A levels on day 2 and day 4. (B) CFSE profiles on day 4 of cells separated into four populations: IL-22⁺IL-17A⁻, IL-22⁺IL-17A⁺, IL-22⁻IL-17A⁺, and IL-22⁻IL-17A⁻. (C) IL-22 expression in naïve DO11 T cells cultured with LPS activated DCs, OVAp, and neutralizing antibodies to either IL-23R or IL-12p40.

FIG. 6. In vivo expression of IL-22 in the absence of IL-6 or IL-23. IL-22 expression in C57BL/6 IL-6^{-/-} (A) and C57BL/6 IL-23p19^{-/-} (B) mice following immunization with OVA. IL-22 expression in wildtype (WT) mice is also shown.

FIG. 7. Flow cytometric and ELISA analysis of in vivo IL-22 co-expression with IL-17A and IL-17F. (A) LN cells stained for CD4 and IL-22, IL-17A, IL-17F, or isotype controls. (B) IL-22 expression in relation to IFN-γ, IL-17A, IL-17F, IL-4, and IL-10 in CD4⁺ T cells. (C) Expression of IL-22 in various IL-17A³⁰ and IL-17F⁺ populations. (D) Expression of IL-17A and IL-17F in IL-22⁺ cells. (E) IL-22 and IL-17A concentrations as determined on day 4 of restimulation by ELISA.

FIG. 8. Analysis of IL-22 production by human Th17 cells and human Th1 cells. (A) IL-22 and IL-17A expression following culture of human CD4⁺ T cells with the indicated cytokines and antibodies. Each line represents an individual donor. (B) The percentage of Th1 or Th17 cells expressing IL-22 were calculated for each of the six donors examined in (A). “Th1 cells” (open bars) were defined by the expression of IFN-γ. “Th17 cells” (stippled bars) were defined by expression of IL-17A.

FIG. 9. Effect of TGF-β on expression of IL-22. (A) IL-22 and IL-17A expression following culture of human CD4⁺ T cells with the indicated cytokines and antibodies. (B) IL-22 expression by naïve CD62L⁺CD4⁺ T cells from DO11.10 mice activated with 1 μg/ml OVAp, and IL-6. Exogenous TGF-β cytokine or a neutralizing antibody to TGF-β was added as indicated.

FIG. 10. IL-22 induces serum amyloid A (SAA) independently of IL-6. (A) SAA serum ELISA following IL-22 injection. (B) Quantitative PCR for SAA1, fibrinogen, haptoglobin, and albumin, normalized to β2 microglobulin, following injection of IL-22. (C) Serum IL-6 and TNF-α ELISAs following IL-22 administration. (D) SAA serum ELISA for C57BL/6 and C57BL/6 IL-6^{-/-} mice administered IL-22.

FIG. 11. Neutrophil numbers and CXCLI levels following IL-22 administration. (A) Neutrophil numbers as determined at the indicated timepoints. (B) CXCL1 proteins levels in serum. (C) Quantitative PCR of CXCL1 transcripts levels in the liver.

FIG. 12. Quantitative PCR analysis of IL-22 and IL-17A or IL-17F induced expression of anti-microbial peptide transcripts. (A) Fold induction of hBD-2, S100A7, S100A8, and S100A9 transcript in primary human keratinocytes treated with IL-22, IL-17A, or IL-17F. (B) Fold induction of hBD-2, S100A7, S100A8, and S100A9 transcript in primary human keratinocytes treated pairwise with combinations of IL-22, IL-17A, and IL-17F.

FIG. 13. IL-22, IL-17A, IL-17F, and IL-23p19 transcript expression in lesional skin of psoriasis patients. (A) Quantitative PCR analysis for IL-22, IL-17A, IL-17F, and IL-23p19. (B) Spearman's rank correlation analysis between IL-22 and IL-17A, IL-22 and IL-17F, IL-17A and IL-17F, IL-22 and IL-23, IL-23 and 117A, and IL-23 and IL-17F.

### DETAILED DESCRIPTION

### I. Definitions

In order that the present invention may be more readily understood, certain terms are first defined. Additional definitions are set forth throughout the detailed description.

The term “antibody” refers to an immunoglobulin or fragment thereof, and encompasses any polypeptide comprising an antigen-binding fragment or an antigen-binding domain. The term includes but is not limited to polyclonal, monoclonal, monospecific, polyspecific, non-specific, humanized, human, single-chain, chimeric, synthetic, recombinant, hybrid, mutated, grafted, and in vitro generated antibodies. Unless preceded by the word “intact”, the term “antibody” includes antibody fragments such as Fab, F(ab′)₂, Fv, scFv, Fd, dAb, and other antibody fragments that retain antigen-binding function. The present invention is not necessarily limited to any particular source, method of production, or other special characteristics of an antibody. Further, the antibodies may be tagged with a detectable or functional label. These labels include radiolabels (e.g., ¹³¹I or ⁹⁹Tc), enzymatic labels (e.g., horseradish peroxidase or alkaline phosphatase), and other chemical moieties (e.g., biotin).

The phrase “inhibit” or “antagonize” cytokine activity and its cognates refer to a reduction, inhibition, or otherwise diminution of at least one activity of that cytokine due to binding an anti-cytokine antibody or soluble receptor to the cytokine or due to competition for binding to the cytokine receptor, wherein the reduction is relative to the activity of cytokine in the in the absence of the same antibody, soluble receptor, or competitive inhibitor. The activity can be measured using any technique known in the art. Inhibition or antagonism does not necessarily indicate a total elimination of cytokine biological activity: A reduction in activity may be about 10%, 20%, 30%, 40%, 50%, 60%, 70%, 80%, 90%, or more.

The term “cytokine activity”, whether used generically or as applied to a particular cytokines such as IL-22, IL-17A, IL-17F, or IL-23, refers to at least one cellular process initiated or interrupted as a result of binding of that cytokine to its receptor(s) on a cell. Cytokine activities for IL-22 include at least one of, but are not limited to: (1) binding to a cellular receptor subunit or complex, such as IL-22R1, IL-10R2, or IL-22R1/IL-10R2; (2) associating with signal transduction molecules (e.g., JAK-1); (3) stimulating phosphorylation of STAT proteins (e.g., STAT5, STAT3, or combination thereof); (4) activating STAT proteins; (5) inducing parameters indicative of an acute phase response, including the modulation of acute phase reactants (e.g., serum amyloid A, fibrinogen, haptoglobin, or serum albumin) and cells (e.g., neutrophils, platelets, or red blood cells; and (6) modulating (e.g., increasing or decreasing) proliferation, differentiation, effector cell function, cytolytic activity, cytokine secretion, survival, or combinations thereof, of epithelial cells, fibroblasts, or immune cells. Epithelial cells include, but are not limited to, cells of the skin, gut, liver, and kidney, as well as endothelial cells. Fibroblasts include, but are not limited to, synovial fibroblasts. Immune cells may include CD8⁺ and CD4⁺ T cells, NK cells, B cells, macrophages, megakaryocytes, and specialized or tissue immune cells, such as those found in inflammed tissues or those expressing an IL-22 receptor.

Cytokine activities for IL-17A and IL-17F include at least one of, but are not limited to: (1) binding to a cellular receptor, such as IL-17R, IL-17A, IL-17RC, IL-17RH1, IL-17RL, IL-17RD, or IL-17RE; (2) inhibition of angiogenesis; (3) modulating (e.g., increasing or decreasing) proliferation, differentiation, effector cell function, cytolytic activity, cytokine secretion, survival, or combinations thereof, of hematopoietic cells or cells present in cartilage, bone, meniscus, brain, kidney, lung, skin and intestine; (4) inducing production of IL-6 and/or IL-8; and (5) stimulating nitric oxide production.

Cytokine activities for IL-23 include at least one of, but are not limited to: (1) binding to a cellular receptor, such as IL-23R or IL-12Rβ1, (2) signaling via Jak2, Tyk2, Stat1, Stat3, Stat4, and Stat5; (3) modulating (e.g., increasing or decreasing) proliferation, differentiation, effector cell function, cytolytic activity, cytokine secretion, survival, or combinations thereof, of immune cells, such as CD4⁺ T cells, NK cells, and macrophages; and (4) inducing production of IL-22, IL-17A, or IL-17F.

The term “isolated” refers to a molecule that is substantially free of its natural environment. For instance, an isolated protein is substantially free of cellular material or other proteins from the cell or tissue source from which it was derived. The term also refers to preparations where the isolated protein is sufficiently pure for pharmaceutical compositions; or at least 70-80% (w/w) pure; or at least 80-90% (w/w) pure; or at least 90-95% pure; or at least 95%, 96%, 97%, 98%, 99%, or 100% (w/w) pure.

The terms “specific binding” or “specifically binds” refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the association constant K_{A} is higher than 10⁶M⁻¹. If necessary, nonspecific binding can be reduced without substantially affecting specific binding by varying the binding conditions. The appropriate binding conditions, such as concentration of antibodies, ionic strength of the solution, temperature, time allowed for binding, concentration of a blocking agent (e.g., serum albumin, milk casein), etc., may be optimized by a skilled artisan using routine techniques.

The term “therapeutic agent” is a substance that treats or assists in treating a medical disorder. As used herein, a therapeutic agent refers to a substance, when administered to a subject along with a composition of the invention, provides a better treatment compared to administration of the therapeutic agent or that inventive composition alone. Non-limiting examples and uses of therapeutic agents are described herein.

The term “effective amount” refers to a dosage or amount that is sufficient to regulate cytokine activity to achieve a desired biological outcome, e.g.,: decreased T cell and/or B cell activity, suppression of autoimmunity, suppression of transplant rejection, suppression of inflammation, systemic or local, etc.

As used herein, a “therapeutically effective amount” refers to an amount which is effective, upon single or multiple dose administration to a subject (such as a human patient) at treating, preventing, curing, delaying, reducing the severity of, ameliorating at least one symptom of a disorder or recurring disorder, or prolonging the survival of the subject beyond that expected in the absence of such treatment.

The term “treatment” refers to a therapeutic or preventative measure. The treatment may be administered to a subject having a medical disorder or who ultimately may acquire the disorder, in order to prevent, cure, delay, reduce the severity of, or ameliorate one or more symptoms of a disorder or recurring disorder, or in order to prolong the survival of a subject beyond that expected in the absence of such treatment.

### II. Modulatory Agents

Various types of agents can be used to regulate or modulate an immune response that is due in part to the activity of one or more of IL-22, IL-17A, IL-17F, or IL-23. In some embodiments, the composition comprises an antibody or antigen-binding fragment thereof that binds to IL-22, an antibody or antigen-binding fragment thereof that binds to IL-17A, an antibody or antigen-binding fragment thereof that binds to IL-17F, an antibody or antigen-binding fragment thereof that binds to IL-23, or a combination of more than one of these antibodies. When the antibody or antigen-binding fragment thereof binds IL-23, it may bind to an eptiope present on the p19 subunit of IL-23, an eptiope present on the p40 subunit of IL-23, or an epitope formed by the combination of the p19 and p40 subunits of IL-23.

In other embodiments, the composition comprises a soluble receptor of IL-22, a soluble receptor of IL-17A, a soluble receptor of IL-17F, a soluble receptor of IL-23, or a combination of these soluble receptors. Examples of soluble receptors include those in which an immunoglobulin Fc domain has been joined to the extracellular portion of the receptor.

In yet other embodiments, the composition comprises a binding protein that binds to IL-22, IL-17A, IL-17F, or IL-23. Examples of binding proteins that bind IL-22 include the naturally-occurring IL-22 binding proteins, such as those described in US2003/0170839, the contents of which are incorporated by reference. When the binding protein binds IL-23, it may bind at a site on the p19 subunit of IL-23, a site on the p40 subunit of IL-23, or a site formed by the combination of the p19 and p40 subunits of IL-23.

In still other embodiments, the composition comprises a combination of 1) one or more antibodies and 2) one or more soluble receptors or binding proteins.

### III. Uses of Modulatory Agents

Compositions that act as agonists or antagonists of one or more of IL-22, IL-17A, IL-17F, or IL-23 can be used to regulate immune responses caused by IL-22 and at least one of IL-17A, IL-17F, and IL-23, such as acting on epithelial cells in solid tissue and indirectly modulating downstream immune responses. Accordingly, antagonist compositions of the invention can be used directly or indirectly to inhibit the activity (e.g., proliferation, differentiation, and/or survival) of an immune or hematopoietic cell (e.g., a cell of myeloid, lymphoid, or erythroid lineage, or precursor cells thereof), and, thus, can be used to treat a variety of immune disorders and hyperproliferative disorders. Non-limiting examples of immune disorders that can be treated include, but are not limited to, autoimmune disorders, e.g., arthritis (including rheumatoid arthritis, juvenile rheumatoid arthritis, osteoarthritis; psoriatic arthritis, lupus-associated arthritis or ankylosing spondylitis), scleroderma, systemic lupus erythematosis, vasculitis, multiple sclerosis, autoimmune thyroiditis, dermatitis (including atopic dermatitis and eczematous dermatitis), myasthenia gravis, inflammatory bowel disease (IBD), Crohn's disease, colitis, diabetes mellitus (type I); inflammatory conditions of, e.g., the skin (e.g., psoriasis), cardiovascular system (e.g., atherosclerosis), nervous system (e.g., Alzheimer's disease), liver (e.g., hepatitis), kidney (e.g., nephritis) and pancreas. (e.g., pancreatitis); cardiovascular disorders, e.g., cholesterol metabolic disorders, oxygen free radical injury, ischemia; disorders associated with wound healing; respiratory disorders, e.g., asthma and COPD (e.g., cystic fibrosis); acute inflammatory conditions (e.g., endotoxemia, septicemia, toxic shock syndrome and infectious disease); transplant rejection and allergy. In one embodiment, the disorder is, an arthritic disorder, e.g., a disorder chosen from one or more of rheumatoid arthritis, juvenile rheumatoid arthritis, osteoarthritis, psoriatic arthritis, or ankylosing spondylitis; a respiratory disorder (e.g., asthma, chronic obstructive pulmonary disease (COPD); or an inflammatory condition of, e.g., the skin (e.g., psoriasis), cardiovascular system (e.g., atherosclerosis), nervous system (e.g., Alzheimer's disease), liver (e.g., hepatitis), kidney (e.g., nephritis), pancreas (e.g., pancreatitis), and gastrointestinal organs, e.g., colitis, Crohn's disease and IBD; acute inflammatory conditions, e.g., endotoxemia, septicemia, toxic shock syndrome and infectious disease; multiple organ failure; respiratory disease (ARD); amyloidosis; nephropathies such as glomerulosclerosis, membranous neuropathy, renal arteriosclerosis, glomerulonephritis, fibroproliferative diseases of the kidney, as well as other kidney disfunctions and renal tumors. Because of IL-22 and IL-17A and IL-17F's effects on epithelia, the compositions and combinations of antagonists described herein can be used to treat epithelial cancers, e.g., carcinoma, melanoma and others.

The cytokines IL-22, IL-17A, IL-17F, and IL-23 are known to be associated with many of these immune disorders and hyperproliferative disorders. Because the expression and activity of these cytokines are now known to be associated with a particular type of CD4 effector T cell and to be inter-dependent upon each other, this invention provides, among other things, methods of treating diseases by administering compositions comprising agents that antagonize the activity of IL-22 and at least one of IL-17A, IL-17F, or IL-23.

One example of a disorder associated with one or more of these cytokines is psoriasis. A study measuring levels of IL-22 and IL-22R1 RNA in paired tissue samples (lesion vs. non-lesion) from human psoriatic patients using quantitative PCR demonstrated that levels of IL-22 and IL-22R1 were upregulated in psoriatic lesions. Other evidence implicates IL-22 in the development of psoriasis. For example, transgenic mice that constitutively express IL-22 present with thick skin, mononuclear immune cell infiltrates, characteristic of psoriatic lesions, and die soon after birth. WO 03/083062. Similarly, administering IL-22 to mice induces thickening of skin and mononuclear immune cell infiltrates. WO 03/083062. IL-22 also induces human keratinocyte hyperplasia, suggesting an important role in skin inflammatory processes. Boniface et al., J. Immunol., (2005) 174:3695-3702. This application also shows, using quantitative PCR in paired tissue samples (lesion vs. non-lesion) from human psoriatic patients, that levels of IL-17A, IL-17F, and IL-23p19 are upregulated in psoriatic lesions. In view of the association of not only IL-22, but also IL-17A and IL-17F, with psoriasis, this application provides methods of treating psoriasis by administering compositions comprising agents that antagonize the activity of IL-22 and at least one of IL-17A, IL-17F, or IL-23p19. Further, because IL-23 is also associated with psoriasis and the studies described in this application demonstrate a key role for IL-23 in maintaining IL-22 expression from Th17 cells, the invention also contemplates administering compositions comprising an IL-23 antagonist and an antagonist of IL-22, optionally with an antagonist of IL-17A or IL-17F.

Another example of a disorder associated with one or more of IL-22, IL-17A, IL-17F, and IL-23 is rheumatoid arthritis (RA). RA is characterized by inflammation in the joints. It is the most frequent form of arthritis, involving inflammation of connective tissue and the synovial membrane, a membrane of the joint. The inflamed synovial membrane often infiltrates the joint and damages joint cartilage and bone. Inhibitors of IL-22 ameliorate symptoms in an animal model of RA (WO 02/068476 A2; U.S. Pat. No. 6,939,545). RA is also associated with IL-23. Recent studies have shown that IL-23p19 deficient mice are resistant to EAE (a model of multiple sclerosis) and collagen-induced arthritis (CIA—a model of RA), demonstrating that IL-23 is an important factor in the pathogenesis of these autoimmune diseases. Mechanistically, this has been attributed to diminished IL-17A and IL-17F expression in IL-23 deficient mice. However, IL-17A deficient mice, while developing less severe disease, are still susceptible to CIA, suggesting that IL-17A does not account for all the functions of IL-23. The studies described in this application demonstrate a key role for IL-23 in maintaining IL-22 expression from Th17 cells. Our data indicate that IL-22, like IL-17A and IL-17F, is downstream of IL-23 signaling in CIA. We have also observed co-expression of IL-22 with IL-17A in CD4 T cells in mice with CIA. Furthermore, in rheumatoid arthritis patients, IL-22 is expressed in synovial tissues and mononuclear cells. Treatment of synovial fibroblasts isolated from patients with IL-22 induced chemokine production (Ikeuchi H. et al. Arthritis Rheum 52:1037-1046). IL-22 also induced IL-6, IL-8, and a variety of chemokines and metallomatrix proteinases from colonic myofibroblasts (Andoh, A. et al. Gastroenterology 129:969-984.). Systemic administration of IL-22 enhanced circulating amounts of serum amelyoid A (SAA), demonstrating that IL-22 can induce parameters indicative of an acute phase response (Dumoutier, L. et al 2000. Proc Natl Acad Sci USA 97:10144-10149.). IL-23p19 transgenic mice also display higher concentrations of circulating SAA (Wiekowski, M. et al. 2001 J Immunol. 166:12 (7563-70), and our data indicate that this effect is at least partially mediated by IL-22.

Accordingly, this application specifically contemplates treating RA using compositions to inhibit not only IL-22, but also one or both of IL-17A and IL-17F. The invention further contemplates administering compositions comprising an antagonist of IL-23 and an antagonist of IL-22, optionally with an antagonist of IL-17A or IL-17F, since IL-23 influences the production of IL-22 and IL-17 from Th17 cells. In addition to treating RA, the methods of this invention may be used to treat other arthritic diseases in humans.

IL-22 is also known to enhance the innate immunity of peripheral tissues by inducing the expression of anti-microbial peptides including beta-defensin 2 (hBD-2), S100A7, S100A8, and S100A9 (Wolk et al., Immunity (2004) 21:241-54; Boniface et al., J. Immunol. (2005) 174:3695-3702). Data in this application indicate that IL-22 and at least one of IL-17A, IL-17F, or IL-23 may be particularly effective in combating microbial infections by inducing expression of one or more anti-microbial peptides, and thus enhancing the innate immune response, because IL-22 can act in cooperation, either additively or synergistically, with IL-17A and IL-17F, and it is induced by IL-23. Accordingly, this application provides methods of inducing an anti-microbial peptide in a mammal in need thereof, comprising administering to the mammal IL-22 and IL-17A, IL-22 and IL-17F, or IL-22, IL-17A, and IL-17F in amounts effective to induce an anti-microbial peptide. In other embodiments, the method of inducing an anti-microbial peptide, in a mammal in need thereof, comprises administering to the mammal IL-22 and IL-23, optionally with IL-17A and/or IL-17F, in amounts effective to induce an anti-microbial peptide. In still other embodiments, the anti-microbial peptide is induced in a cell, such as a keratinocyte.

An acute phase response is a collection of biochemical, physiologic, and behavioral changes indicative of an inflammatory condition. The modulation of specific proteins known as acute phase reactants is a biochemical hallmark of an acute phase response and of inflammation. (Reviewed in Gabay & Kushner, N. Engl. J. Med. (1999) 340:448-55.) The concentration of some acute-phase proteins typically increase in response to inflammation. Examples of those proteins include C-reactive protein, serum amyloid A, fibrinogen, and haptoglobin. The concentration of other proteins, such as albumin, transferrin, and α-fetoprotein, typically decrease in the acute phase response. The pattern of expression of acute phase proteins can vary depending upon the underlying condition, and the pattern of expression and the relative levels of the various acute phase proteins can be used to determine the nature and severity of the inflammation. Data in this application indicate that IL-22 can effect an acute phase response. Accordingly, this application provides methods of inducing or enhancing the acute phase response by administering IL-22 and at least one of IL-17A, IL-17F, or IL-23. In another embodiment the application provides methods of increasing the expression of an acute phase protein, such as C-reactive protein, serum amyloid A, fibrinogen, or haptoglobin, or decreasing the expression of an acute phase protein, such as albumin, transferrin, or α-fetoprotein, by administering IL-22 and at least one of IL-17A, IL-17F, or IL-23. The application further contemplates administering compositions comprising an antagonist of IL-22, optionally with an antagonist of one or more of IL-17A, IL-17F, or IL-23 to reduce or inhibit the acute phase response. In another embodiment the application provides methods of increasing the expression of an acute phase protein, such as C-reactive protein, serum amyloid A, fibrinogen, or haptoglobin, or decreasing the expression of an acute phase protein, such as albumin, transferrin, or α-fetoprotein, by administering an antagonist of IL-22, optionally with an antagonist of one or more of IL-17A, IL-17F, or IL-23.

### IV. Combination Therapy Comprising Additional Therapeutic Agents

Although the application includes compositions comprising combinations of agents that inhibit the activity of IL-22 and at least one of IL-17A, IL-17F, or IL-23, these compositions may further comprise one or more additional therapeutic agents that are advantageous for treating various diseases. The term “in combination” in this context means that the composition comprising the therapeutic agents is given substantially contemporaneously, either simultaneously or sequentially, with the composition comprising a combination of agents that inhibit the activity of one or more of IL-22, IL-17A, IL-17F, or IL-23. In one embodiment, if given sequentially, at the onset of administration of the second composition, the first of the two compositions is still detectable at effective concentrations at the site of treatment. In another embodiment, if given sequentially, at the onset of administration of the second composition, the first of the two compositions is not detectable at effective concentrations at the site of treatment.

For example, the combination therapy can include a composition comprising at least one anti-IL-22 antibody and at least one anti-IL-17A, anti-IL-17F, or anti-IL-23 antibody co-formulated with, and/or co-administered with, at least one additional therapeutic agent. The additional therapeutic agent may include at least one inhibitor of a cytokine other than IL-22, IL-17A, IL-17F, or IL-23; a growth factor inhibitor; an immunosuppressant an anti-inflammatory agent; a metabolic inhibitor; an enzyme inhibitor; a cytotoxic agent; and a cytostatic agent, as described in more detail below. The compositions and combinations of the invention can be used to regulate inflammatory conditions associated with IL-22 and at least one of IL-17A, IL-17F, or IL-23, e.g., by modulating cytokine signaling through receptors located on fibrobalsts and/or epithelial cells of a variety of tissues, including, but not limited to, those of the pancreas, skin, lung, gut, liver, kidney, salivary gland, and vascular endothelia, in addition to potentially activated and tissue localized immune cells.

In one embodiment, the additional therapeutic agent is a standard treatment for arthritis, including, but not limited to, non-steroidal anti-inflammatory agents (NSAIDs); corticosteroids, including prednisolone, prednisone, cortisone, and triamcinolone; and disease modifying anti-rheumatic drugs (DMARDs), such as methotrexate, hydroxychloroquine (Plaquenil™) and sulfasalazine, leflunomide (Arava™), tumor necrosis factor inhibitors, including etanercept (Enbrel™), infliximab (Remicade™) (with or without methotrexate), and adalimumab (Humira™), anti-CD20 antibody (e.g., Rituxan™), soluble interleukin-1 receptor, such as anakinra (Kineret™), gold, minocycline (Minocin™), penicillamine, and cytotoxic agents, including azathioprine, cyclophosphamide, and cyclosporine. Such combination therapies may advantageously utilize lower dosages of the administered therapeutic agents, thus avoiding possible toxicities or complications associated with the various monotherapies. Moreover, the therapeutic agents disclosed are expected to provide enhanced and/or synergistic effects.

The additional therapeutic agents may be those agents that interfere at different stages in the autoimmune and subsequent inflammatory response. In one embodiment, the composition comprising a combination of agents that inhibit the activity of one or more of IL-22, IL-17A, IL-17F, or IL-23 may be co-formulated with, and/or co-administered with, at least one growth factor antagonist or an antagonist of a cytokine other than IL-22, IL-17A, IL-17, or IL-23. The antagonists may include soluble receptors, peptide inhibitors, small molecules, ligand fusions, antibodies (that bind cytokines or growth factors or their receptors or other cell surface molecules) and binding fragments thereof, and “anti-inflammatory cytokines” and agonists thereof.

Non-limiting examples of the additional therapeutic agents include, but are not limited to, antagonists of at least one interleukin (e.g., IL-1, IL-2, IL-6, IL-7, IL-8, IL-12 (or one of its subunits p35 or p40), IL-13, IL-15, IL-16, IL-18, IL-19, IL-20, IL-21, IL-24, IL-26, IL-28, IL-29, IL-31, and IL-33); cytokine (e.g., TNFα, LT, EMAP-II, and GM-CSF); and growth factor (e.g., FGF and PDGF). The agents may also include, but are not limited to, antagonists of at least one receptor for an interleukin, cytokine, and growth factor. Inhibitors (e.g., antibodies) of cell surface molecules such as CD2, CD3, CD4, CD8, CD20 (e.g. Rituxan™), CD25, CD28, CD30, CD40, CD45, CD69, CD80 (B7.1), CD86 (B7.2), CD90, or their ligands (e.g., CD154 (gp39, CD40L)), or LFA-1/ICAM-1 and VLA-4/VCAM-1 (Yusuf-Makagiansar et al., Med. Res. Rev., (2002) 22 (2):146-67)) can also be employed as additional therapeutic agents. In certain embodiments, antagonists that can be used as additional therapeutic agents may include antagonists of IL-1, IL-12 (or one of its subunits p35 or p40), TNFα, IL-15, IL-18, IL-19, IL-20, and IL-21, and their receptors.

Examples of those agents include IL-12 antagonists (such as antibodies that bind IL-12 (see e.g., WO 00/56772) or one of its subunits p35 or p40); IL-12 receptor inhibitors (such as antibodies to the IL-12 receptor); and soluble IL-12 receptor and fragments thereof. Examples of IL-15 antagonists include antibodies against IL-15 or its receptor, soluble fragments of the IL-15 receptor, and IL-15-binding proteins. Examples of IL-18 antagonists include antibodies to IL-18, soluble fragments of the IL-18 receptor, and IL-18 binding proteins (IL-18BP, Mallet et al., Circ. Res., (2001) 28). Examples of IL-1 antagonists include Interleukin-1-Converting Enzyme (ICE) inhibitors (such as Vx740), IL-1 antagonists (e.g., IL-1RA (ANIKINRA (or Kineret™), AMGEN)), sIL-1RII (Immunex), and anti-IL-1 receptor antibodies.

Examples of TNF antagonists include antibodies to TNF (e.g., human TNFα), such as D2E7 (human anti-TNFα antibody, U.S. Pat. No. 6,258,562, Humira™, BASF); CDP-571/CDP-870/BAY-10-3356 (humanized anti-TNFα antibodies, Celltech/Pharmacia); cA2 (chimeric anti-TNFα antibody, Remicade™, Centocor); and anti-TNF antibody fragments (e.g., CPD870). Other examples include soluble TNF receptor (e.g., human p55 or p75) fragments and derivatives thereof, such as p55 kdTNFR-IgG (55 kD TNF receptor-IgG fusion protein, Lenercept™) and 75 kdTNFR-IgG (75 kD TNF receptor-IgG fusion protein, Enbrel™, Immunex, see, e.g., Arthritis & Rheumatism, (1994) Vol. 37, S295; J. Invest. Med., (1996) Vol. 44, 235A). Further examples include enzyme antagonists (e.g., TNFα converting enzyme inhibitors (TACE) such as alpha-sulfonyl hydroxamic acid derivative (WO 01/55112) or N-hydroxyformamide inhibitor (GW 3333, -005, or -022)) and TNF-bp/s-TNFR (soluble TNF binding protein, see e.g., Arthritis & Rheumatism (1996) Vol. 39, No. 9 (supplement), S284; and Am. J. Physiol. Heart Circ. Physiol. (1995) Vol. 268, pp. 37-42). TNF antagonists may be soluble TNF receptor (e.g., human p55 or p75) fragments and derivatives, such as 75 kdTNFR-IgG; and TNFα converting enzyme (TACE). inhibitors.

In other embodiments, the composition comprising a combination of agents that inhibit the activity of one or more of IL-22, IL-17A, IL-17F, or IL-23 can be administered in combination with at least one of the following: IL-13 antagonists, such as soluble IL-13 receptors and/or anti-IL-13 antibodies; and IL-2 antagonists, such as IL-2 fusion proteins (e.g., DAB 486-IL-2 and/or DAB 389-IL-2, Seragen, see e.g., Arthritis & Rheumatism, (1993) Vol. 36, 1223) and anti-IL-2R antibodies (e.g., anti-Tac (humanized antibody, Protein Design Labs, see Cancer Res., (1990) 50 (5):1495-502)). Another additional therapeutic agent that can be combined with a composition comprising a combination of agents that inhibit the activity of one or more of IL-22, IL-17A, IL-17F, or IL-23 is non-depleting anti-CD4 inhibitors such as IDEC-CE9.1/SB 210396 (anti-CD4 antibody, IDEC/SmithKline). Yet other additional therapeutic agents that can be combined with a composition comprising a combination of agents that inhibit the activity of one ore more of IL-22, IL-17A, IL-17F, or IL-23 include antagonists (such as antibodies, soluble receptors, or antagonistic ligands) of costimulatory molecules, such as CD80 (B7.1) and CD86 (B7.2); ICOSL, ICOS, CD28, and CTLA4 (e.g., CTLA4-Ig (atabacept)); P-selectin glycoprotein ligand (PSGL); and anti-inflammatory cytokines and agonists thereof (e.g., antibodies). The anti-inflammatory cytokines may include IL-4 (DNAX/Schering); IL-10 (SCH 52000, recombinant IL-10, DNAX/Schering); IL-13; and TGF.

In other embodiments, the additional therapeutic agent that can be combined with a composition comprising a combination of agents that inhibit the activity of one ore more of IL-22, IL-17A, IL-17F, or IL-23 is at least one anti-inflammatory drug, immunosuppressant, metabolic inhibitor, and enzymatic inhibitor. Non-limiting examples of such drugs or inhibitors include, but are not limited to, at least one of: non-steroidal anti-inflammatory drug (NSAID) (such as ibuprofen, Tenidap (see e.g., Arthritis & Rheumatism, (1996) Vol. 39, No. 9 (supplement), S280)), Naproxen (see e.g., Neuro Report, (1996) Vol. 7, pp. 1209-1213), Meloxicam, Piroxicam, Diclofenac, and Indomethacin); Sulfasalazine (see e.g., Arthritis & Rheumatism (1996) Vol. 39, No. 9 (supplement), S281); corticosteroid (such as prednisolone); cytokine suppressive anti-inflammatory drug (CSAID); and an inhibitor of nucleotide biosynthesis (such as an inhibitor of purine biosynthesis (e.g., folate antagonist such as methotrexate)) or an inhibitor of pyrimidine biosynthesis (e.g., a dihydroorotate dehydrogenase (DHODH), such as leflunomide (see e.g., Arthritis & Rheumatism, (1996) Vol. 39, No. 9 (supplement), S131; Inflammation Research, (1996) Vol. 45, pp. 103-107)).

Examples of additional inhibitors include at least one of: corticosteroid (oral, inhaled and local injection); immunosuppressant (such as cyclosporin and tacrolimus (FK-506)); a mTOR inhibitor (such as sirolimus (rapamycin) or a rapamycin derivative (e.g., ester rapamycin derivative such as CCI-779 (Elit. L., Current Opinion Investig. Drugs, (2002) 3 (8):1249-53; Huang, S. et al., Current Opinion Investig. Drugs (2002) 3 (2):295-304))); an agent which interferes with the signaling of proinflammatory cytokines such as TNFα and IL-1 (e.g., IRAK, NIK, IKK, p38 or a MAP kinase inhibitor); a COX2 inhibitor (e.g., celecoxib and variants thereof (MK-966), see e.g., Arthritis & Rheumatism, (1996) Vol. 39, No. 9 (supplement), S81); a phosphodiesterase inhibitor (such as R973401, see e.g., Arthritis & Rheumatism, (1996) Vol. 39, No. 9 (supplement), S282)); a phospholipase inhibitor (e.g., an inhibitor of cytosolic phospholipase 2 (cPLA2) such as trifluoromethyl ketone analogs (U.S. Pat. No. 6,350,892)); an inhibitor of vascular endothelial cell growth factor (VEGF); an inhibitor of the VEGF receptor; and an inhibitor of angiogenesis.

The composition comprising a combination of agents that inhibit the activity of IL-22 and at least one of IL-17A, IL-17F, or IL-23 disclosed herein can be used in combination with additional therapeutic agents to treat specific immune disorders as discussed in further detail below.

Non-limiting examples of additional therapeutic agents for treating arthritic disorders (e.g., rheumatoid arthritis, inflammatory arthritis, rheumatoid arthritis, juvenile rheumatoid arthritis, osteoarthritis and psoriatic arthritis) include at least one of the following: TNF antagonists (such as anti-TNF antibodies); soluble fragments of TNF receptors (e.g., human p55 and p75) and derivatives thereof (such as p55 kdTNFR-IgG (55 kD TNF receptor-IgG fusion protein, Lenercept™) and 75 kdTNFR-IgG (75 kD TNF receptor-IgG fusion protein, Enbrel™)); TNF enzyme antagonists (such as TACE inhibitors); antagonists of IL-12 (or one of its subunits p35 or p40), IL-15, IL-18, IL-19, IL-20, IL-21, and IL-24; T cell and B cell depleting agents (such as anti-CD4, anti-CD20, or anti-CD22 antibodies); small molecule inhibitors (such as methotrexate and leflunomide); sirolimus (rapamycin) and analogs thereof (e.g., CCI-779); Cox-2 and cPLA2 inhibitors; NSAIDs; p38, TPL-2, Mk-2, and NFκB inhibitors; RAGE or soluble RAGE; P-selectin or PSGL-1 inhibitors (such as small molecule inhibitors and antibodies to); estrogen receptor beta (ERB) agonists, and ERB-NFκB antagonists.

Non-limiting examples of additional therapeutic agents for treating multiple sclerosis include interferon-β for example, IFNβ-1a and IFNβ-1b), copaxone, corticosteroids, IL-1 inhibitors, TNF inhibitors, antibodies to CD40 ligand, antibodies to CD80, and IL-12 antagonists.

Non-limiting examples of additional therapeutic agents for treating inflammatory bowel disease or Crohn's disease include budenoside; epidermal growth factor; corticosteroids; cyclosporine; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 monoclonal antibodies; anti-IL-6 monoclonal antibodies; growth factors; elastase inhibitors; pyridinyl-imidazole compounds; TNF antagonists as described herein; IL-4, IL-10, IL-13, and/or TGFβ or agonists thereof (e.g., agonist antibodies); IL-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of Platelet Activating Factor (PAF); ciprofloxacin; and lignocaine.

Non-limiting examples of additional therapeutic agents for regulating immunue responses, e.g., treating or inhibiting transplant rejection and graft-versus-host disease, include the following: antibodies against cell surface molecules, including but not limited to CD25 (IL-2 receptor a), CD11a (LFA-1), CD54 (ICAM-1), CD4, CD45, CD28/CTLA4, CD80 (B7-1), CD86 (B7-2), or combinations thereof, and general immunosuppressive agents, such as cyclosporin A or FK506.

Another aspect of the present invention accordingly relates to kits for carrying out the administration of a composition comprising a combination of agents that inhibit the activity of IL-22 and at least one of IL-17A, IL-17F, or IL-23, optionally with additional therapeutic agents. In one embodiment, the kit comprises a composition comprising an IL-22 antagonist, and an antagonist of at least one of IL-17A, IL-17F, or IL-23 formulated in a pharmaceutical carrier. The kit may further comprise at least one additional therapeutic agent, formulated as appropriate in one or more separate pharmaceutical preparations.

### V. Pharmaceutical Compositions and Methods of Administration

Certain methods described in this application utilize compositions suitable for pharmaceutical use and administration to patients. These compositions comprise a pharmaceutical excipient and one or more antibodies, one or more soluble receptors, one or more binding proteins, or combinations of those antibodies, soluble receptors, and/or binding proteins. As used herein, “pharmaceutical excipient” includes solvents, dispersion media, coatings, antibacterial and antifungal agents, isotonic and absorption delaying agents, etc., that are compatible with pharmaceutical administration. Use of these agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition can be formulated to be compatible with its intended route of administration. Methods to accomplish the administration are known to those of ordinary skill in the art. It may also be possible to create compositions which may be topically or orally administered, or which may be capable of transmission across mucous membranes. For example, the administration may be intravenous, intraperitoneal, intramuscular, intracavity, subcutaneous, cutaneous, or transdermal.

Solutions or suspensions used for intradermal or subcutaneous application typically include at least one of the following components: a sterile diluent such as water, saline solution, fixed oils, polyethylene glycol, glycerine, propylene glycol, or other synthetic solvent; antibacterial agents such as benzyl alcohol or methyl parabens; antioxidants such as ascorbic acid or sodium bisulfite; chelating agents such as ethylenediaminetetraacetic acid (EDTA); buffers such as acetate, citrate, or phosphate; and tonicity agents such as sodium chloride or dextrose. The pH can be adjusted with acids or bases. Such preparations may be enclosed in ampoules, disposable syringes; or multiple dose vials.

Solutions or suspensions used for intravenous administration include a carrier such as physiological saline, bacteriostatic water, Cremophor EL™ (BASF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent which delays absorption, e.g., aluminum monostearate and gelatin.

Oral compositions include an inert diluent or edible carrier. The composition can be enclosed in gelatin or compressed into tablets. For the purpose of oral administration, the antibodies can be incorporated with excipients and placed in tablets, troches, or capsules. Pharmaceutically compatible binding agents or adjuvant materials can be included in the composition. The tablets, troches, and capsules, may contain (1) a binder such as microcrystalline cellulose, gum tragacanth or gelatin; (2) an excipient such as starch or lactose, (3) a disintegrating agent such as alginic acid, Primogel, or corn starch; (4) a lubricant such as magnesium stearate; (5) a glidant such as colloidal silicon dioxide; or (6) a sweetening agent or a flavoring agent.

The pharmaceutical composition may also be administered by a transmucosal or transdermal route. For example, antibodies that comprise a Fc portion may be capable of crossing mucous membranes in the intestine, mouth, or lungs (via Fc receptors). Transmucosal administration can be accomplished through the use of lozenges, nasal sprays, inhalers, or suppositories. Transdermal administration can also be accomplished through the use of a composition containing ointments, salves, gels, or creams known in the art. For transmucosal or transdermal administration, penetrants appropriate to the barrier to be permeated are used. For administration by inhalation, the antibodies are delivered in an aerosol spray from a pressured container or dispenser, which contains a propellant (e.g., liquid or gas) or a nebulizer.

In certain embodiments, the pharmaceutical compositions are prepared with carriers to protect the active component against rapid elimination from the body. Biodegradable polymers (e.g., ethylene vinyl acetate, polyanhydrides, polyglycolic acid, collagen, polyorthoesters, polylactic acid) are often used. Methods for the preparation of such formulations are known by those skilled in the art. Liposomal suspensions can be used as pharmaceutically acceptable carriers too. The liposomes can be prepared according to established methods known in the art (U.S. Pat. No. 4,522,811).

The pharmaceutical compositions are administered in therapeutically effective amounts as described. Therapeutically effective amounts may vary with the subject's age, condition, sex, and severity of medical condition. Appropriate dosage may be determined by a physician based on clinical indications. The compositions may be given as a bolus dose to maximize the circulating levels of active component of the composition for the greatest length of time. Continuous infusion may also be used after the bolus dose.

As used herein, the term “subject” is intended to include human and non-human animals. The term “non-human animals” of the invention includes all vertebrates, such as non-human primates, sheep, dogs, cows, chickens, amphibians, reptiles, etc.

Examples of dosage ranges that can be administered to a subject can be chosen from: 1 μg/kg to 20 mg/kg, 1 μg/kg to 10 mg/kg, 1 μg/kg to 1 mg/kg, 10 μg/kg to 1 mg/kg, 10 μg/kg to 100 μg/kg, 100 μg/kg to 1 mg/kg, 250 μg/kg to 2 mg/kg, 250 μg/kg to 1 mg/kg, 500 μg/kg to 2 mg/kg, 500 μg/kg to 1 mg/kg, 1 mg/kg to 2 mg/kg, 1 mg/kg to 5 mg/kg, 5 mg/kg to 10 mg/kg, 10 mg/kg to 20 mg/kg, 15 mg/kg to 20 mg/kg, 10 mg/kg to 25 mg/kg, 15 mg/kg to 25 mg/kg, 20 mg/kg to 25 mg/kg, and 20 mg/kg to 30 mg/kg (or higher). These dosages may be administered daily, weekly, biweekly, monthly, or less frequently, for example, biannually, depending on dosage, method of administration, disorder or symptom(s) to be treated, and individual subject characteristics. Dosages can also be administered via continuous infusion (such as through a pump). The administered dose may also depend on the route of administration. For example, subcutaneous administration may require a higher dosage than intravenous administration.

In certain circumstances, it may be advantageous to formulate compositions in dosage unit form for ease of administration and uniformity of dosage. Dosage unit form as used herein refers to physically discrete units suited for the patient. Each dosage unit contains a predetermined quantity of antibody calculated to produce a therapeutic effect in association with the carrier. The dosage unit depends on the characteristics of the antibodies and the particular therapeutic effect to be achieved.

Toxicity and therapeutic efficacy of the pharmaceutical composition can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅/ED₅₀.

The data obtained from the cell culture assays and animal studies can be used to formulate a dosage range in humans. The dosage of these compounds may lie within the range of circulating antibody concentrations in the blood, which includes an ED₅₀ with little or no toxicity. The dosage may vary within this range depending upon the dosage composition form employed and the route of administration. The therapeutically effective dose can be estimated initially using cell culture assays. A dose may be formulated in animal models to achieve a circulating plasma concentration range that includes the IC₅₀ (i.e., the concentration of agent which achieves a half-maximal inhibition of symptoms). The effects of any particular dosage can be monitored by a suitable bioassay. Examples of suitable bioassays include DNA replication assays, transcription-based assays, receptor-binding assays, and other immunological assays.

### VI. Diagnostic Uses

The antagonists may also be used to detect the presence of IL-22, and at least one of IL-17A, IL-17F, or IL-23 in a biological sample. These cytokines can be detected either extracellularly or intracellularly using methods known in the art, including the methods disclosed in this application. By correlating the presence or level of these proteins with a medical condition, one of skill in the art can diagnose the associated medical condition. For example, IL-22 induces changes associated with those caused by inflammatory cytokines (such as IL-1 and TNFα), and inhibitors of IL-22 ameliorate symptoms in an animal model of rheumatoid arthritis (WO 02/068476 A2). As disclosed in this application, IL-22 is co-expressed with IL-17A and IL-17F in psoriatic lesions and functions in synergy with those cytokines to enhance the expression of anti-microbial peptides. Therefore, illustrative medical conditions that may be diagnosed in accordance with this disclosure include psoriasis and rheumatoid arthritis. Multiple sclerosis, inflammatory bowel disease, and Crohn's disease can also be diagnosed in accordance with this application. Further, since this application shows that IL-22 can induce an acute-phase response, that response can be monitored using methods in accordance with the disclosure.

Antibody-based detection methods are well known in the art, and include ELISA, radioimmunoassays, immunoblots, Western blots, flow cytometry, immunofluorescence, immunoprecipitation, and other related techniques. The antibodies may be provided in a diagnostic kit. The kit may contain other components, packaging, instructions, or other material to aid the detection of the protein and use of the kit.

Antibodies may be modified with detectable markers, including ligand groups (e.g., biotin), fluorophores and chromophores, radioisotopes, electron-dense reagents, or enzymes. Enzymes are detected by their activity. For example, horseradish peroxidase is detected by its ability to convert tetramethylbenzidine (TMB) to a blue pigment, quantifiable with a spectrophotometer. Other suitable binding partners include biotin and avidin, IgG and protein A, and other receptor-ligand pairs known in the art.

Antibodies can also be functionally linked (e.g., by chemical coupling, genetic fusion, non-covalent association or otherwise) to at least one other molecular entity, such as another antibody (e.g., a bispecific or a multispecific antibody), toxins, radioisotopes, cytotoxic or cytostatic agents, among others. Other permutations and possibilities are apparent to those of ordinary skill in the art, and they are considered equivalents within the scope of this invention.

When the detection method is an in vitro method, it includes: (1) contacting the sample or a control sample with a first reagent that binds to IL-22 and a second reagent that binds to IL-17A, IL-17F, or IL-23, and (2) detecting formation of a complex between the first and second reagents and the sample or the control sample, wherein a statistically significant change in the formation of the complex in the sample relative to a control sample, is indicative of the presence of the cytokines in the sample. In one embodiment, the method includes contacting a sample comprising cells with a labeled regeant, such as a fluorescent antibody, that binds to IL-22, IL-17A, IL-17F, or IL-23 within the cells. The amount of reagent detected within a cell is directly proportional to the amount of intracellular IL-22, IL-17A, IL-17F, or IL-23 expressed within the cell.

The detection method can also be an in vivo detection method (e.g., in vivo imaging in a subject). The method can be used to diagnose a disorder, e.g., a disorder as described herein. The method includes: (1) administering a first reagent that binds to IL-22 and a second reagent that binds to IL-17A, IL-17F, or IL-23 to a subject or a control subject under conditions that allow binding of the first and second reagents to their cytokines, and (2) detecting formation of a complex between the first and second reagents and their cytokines, wherein a statistically significant change in the formation of the complex in the subject relative to a control, e.g., a control subject, is indicative of the presence of the cytokines.

### EXAMPLES

### Example 1

IL-22 transcript is more highly expressed in Th17 cells than in Th1 or Th2 cells.

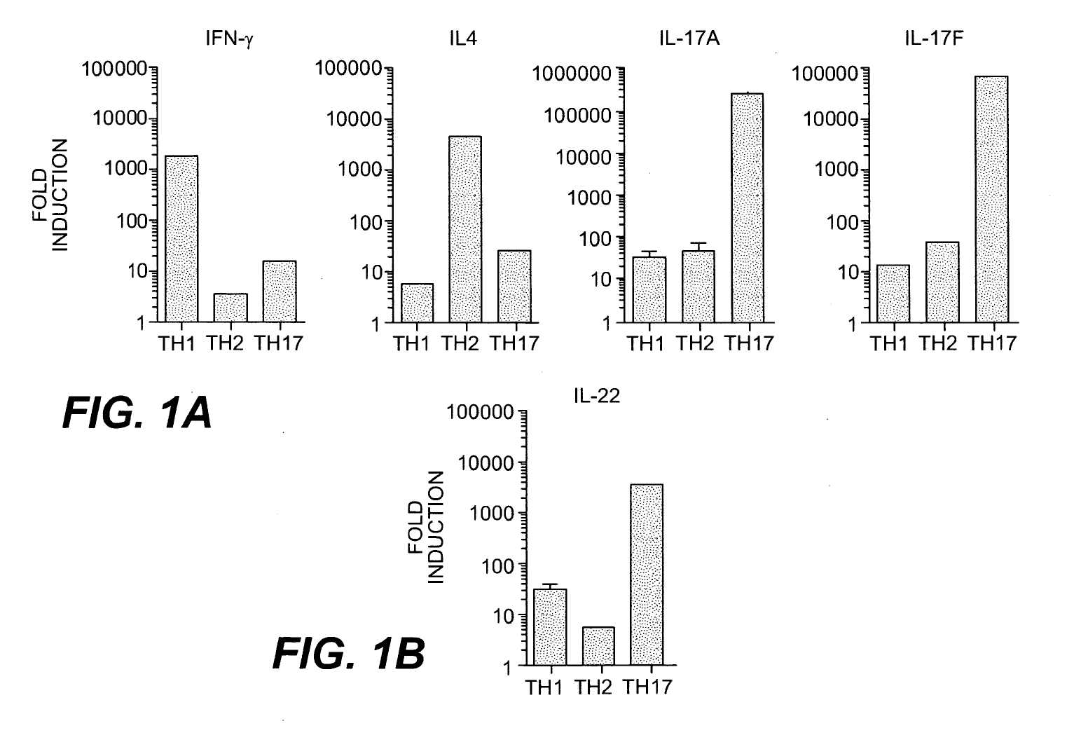

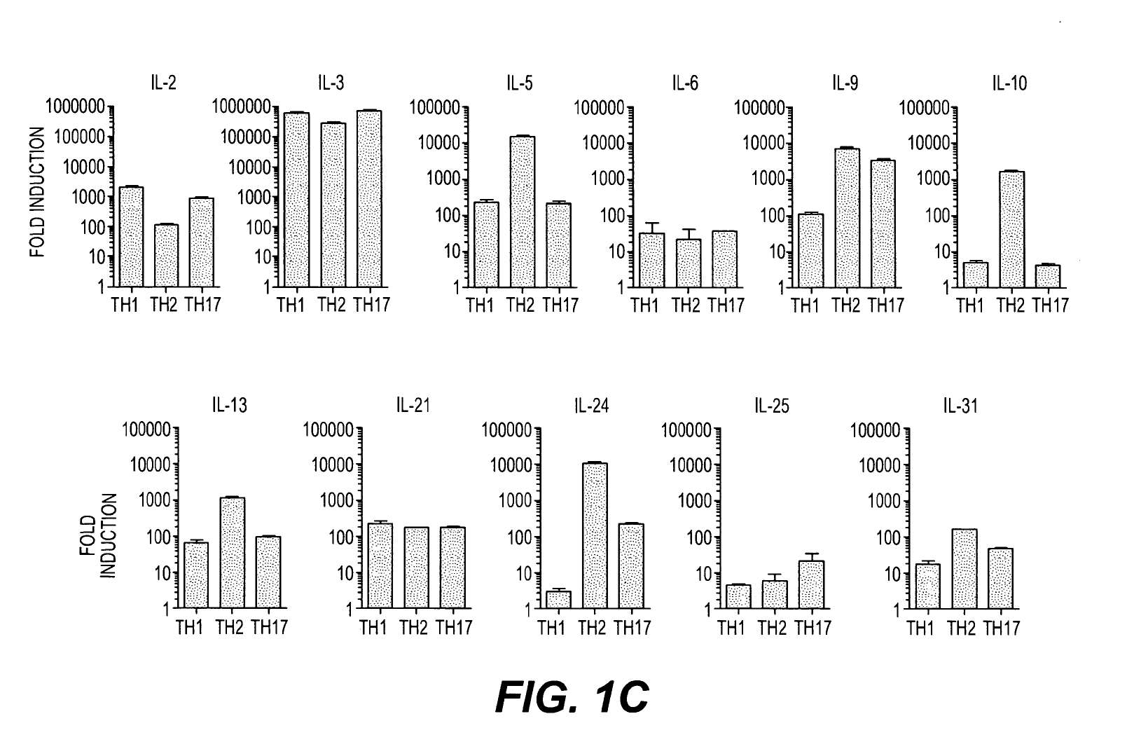

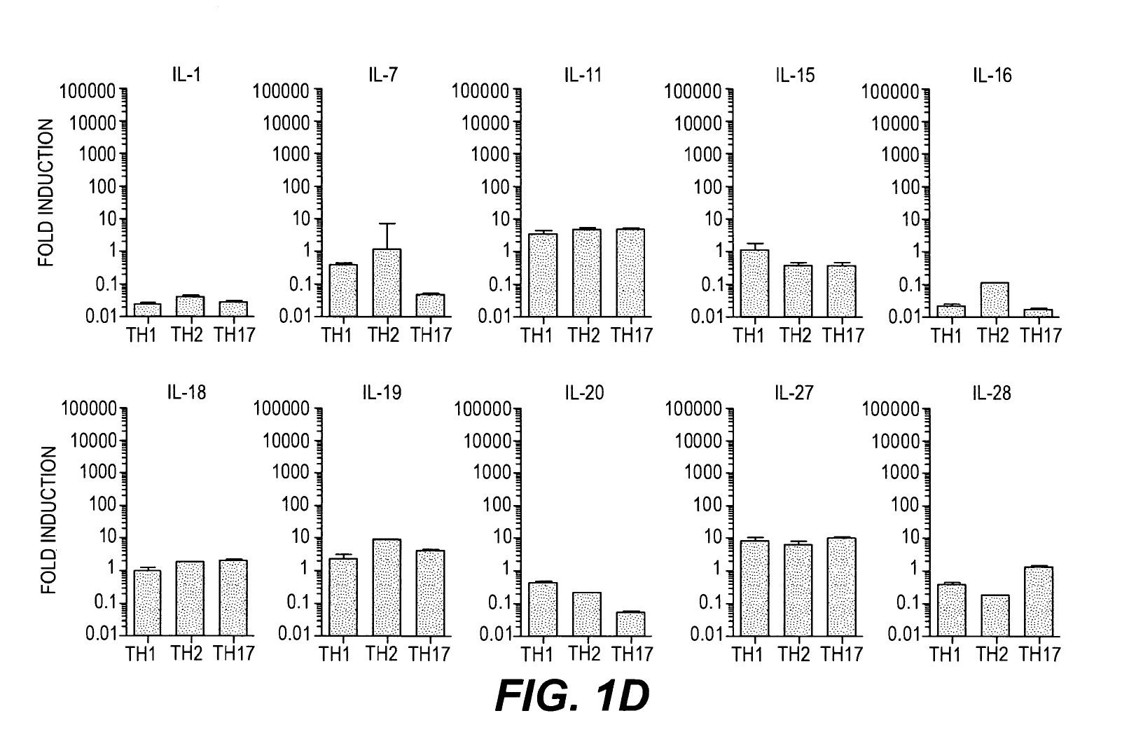

Th17 cells are thought to produce IL-17A and IL-17F in a lineage specific manner. In order to identify other potential Th17 cytokines, naïve (CD62L^{Hi}CD4⁺) T cells purified from C.Cg-Tg(DO11.10)10DIo TCR transgenic mice (Jackson Laboratories) were differentiated to the Th1 (IL-12, anti-IL-4), Th2 (IL-4, anti-IFN-γ), and Th17 (TGF-β, IL-6, IL-1β, TNF-α, IL-23, anti-IFN-γ, and anti-IL-4) lineages. Naïve (CD62L^{Hi}CD4⁺) T cells were purified from spleens of DO11 mice by CD4 negative selection followed by CD62L positive selection according to the manufacturer's directions (Miltenyi Biotec). All lymphocyte cultures were grown in RPMI 1640 supplemented with 10% FBS, 2 mM L-glutamine, 5 mM HEPES, 100 U/ml Pen-Strep, and 2.5 μM β-mercaptoethanol. Purity of CD4⁺CD62L^{Hi} cells was above 98%. 2×10⁵ DO11 T cells were cultured with 4×10⁶ irradiated BALB/cByJ splenocytes (3300 rad) and 1 μg/ml OVA₃₂₃₋₃₃₉ peptide (OVAp) (New England Peptide). Recombinant cytokines were used at 10 ng/ml, except for IL-4 (1 ng/ml) and TGF-β (20 ng/ml). Neutralizing antibodies were used at 10 μg/ml. Murine IL-4, IL-6, IL-12, IL-23, and TNF-α were purchased from R&D Systems. TGF-β was purchased from Sigma. IL-1β was obtained from Bender Medsystems. Antibodies to IFN-γ (XMG1.2) and IL-4 (BVD4-1D11) were purchased from Pharmingen. After differentiating for 7 days, CD4 T cells were re-purified and rested overnight. Cells were then restimulated with 50 ng/ml PMA, 1 μg/ml ionomycin, and with the following conditions: Th1 cells (IL-12, anti-IL-4), Th2 cells (IL-4, anti-IFN-γ), or Th17 (IL-23, anti-IFN-γ, anti-IL-4) for 6 hrs. The expression of cytokines after restimulation were then examined by quantitative PCR. RNA was prepared and quantitative PCR for cytokine transcripts was performed using SYBR Green Platinum Taq (Invitrogen) and pre-qualified primers (Qiagen). All cytokine concentrations were normalized to HPRT. Fold induction was calculated using the ΔΔCt method relative to purified, unactivated naïve DO11 T cells. Data shown in FIG. 1 are average±SD and are representative of two experiments.

Th1 cells expressed the highest amounts of IFN-γ transcript, Th2 cells had the highest abundance of IL-4, and Th17 cells produced the greatest abundance of IL-17A and IL-17F, demonstrating that these cells were successfully differentiated (FIG. 1A). Of 22 additional interleukins examined, IL-22 transcript was higher in Th17 cells relative to Th1 cells by ˜120 fold and relative to Th2 cells by ˜700 fold (FIG. 1B). In contrast, expression of IL-2, IL-3, IL-5, IL-6, IL-9, IL-10, IL-13, IL-21, IL-24, IL-25, and IL-31 was equivalent or more abundant in Th1 or Th2 cells compared to Th17 (FIG. 1C). Other cytokines including IL-1, IL-7, IL-11, IL-15, IL-16, IL-18, IL-19, IL-20, IL-27, and IL-28 were not expressed highly in any of the T cell lineages (FIG. 1D). Therefore, IL-22 transcript was identified as one of 22 interleukin transcripts examined that is expressed at higher amounts by Th17 cells than by Th1 or Th2.

### Example 2

Th17 cells are the main producers of IL-22.

IL-22 is a member of the IL-10 family, along with IL-10, IL-19, IL-20, IL-24, and IL-26 (Dumoutier et al., J Immunol, (2000) 164:1814-19; Xie et al., J. Biol. Chem. (2000) 275:31335-39; Renauld et al., Nat. Rev. Immunol. (2003) 3:667-76; Pestka et al., Ann. Rev. Immunol. (2004) 22:929-79). Members of this family share strong structural homology with IL-10. Human IL-22 is located on chromosome 12q15 (mouse chromosome 10), approximately 90 kb away from the IFN-γ locus. Previous reports have demonstrated that activation of human CD4 T cells with IL-12 and anti-IL-4 enhanced IL-22 transcript expression, suggesting that Th1 cells express IL-22 (Wolk et al., J. Immunol. (2002) 168:5397-402; Gurney, A. L., Int. Immunopharmacol. (2004) 4:669-677). However, the expression of IL-22 protein from T cells has not been reported.

To examine IL-22 protein expression, monoclonal antibodies (Ab-01, Ab-02, Ab-03) to murine IL-22 were generated using methods similar to those described previously (Li et al., Int. Immunopharmacol. (2004) 4:693-708) and IL-22 protein concentrations were determined by ELISA. Naïve DO11 T cells were activated with irradiated splenocytes, 1 μg/ml OVAp, and various cytokines and antibodies as indicated. Murine IL-4, IL-6, IL-12, IL-23, and TNF-α, were purchased from R&D Systems. TGF-β was purchased from Sigma. Murine IL-1β was obtained from Bender Medsystems. IL-22 and IL-17F were generated by methods as previously described (Li et al., Int. Immunopharmacol. (2004) 4:693-708). Antibodies to IFN-γ (XMG1.2), IL-4 (BVD4-1D11), IL-17A (TC11-18H10), and CD4 (RM4-5) were purchased from Pharmingen. Anti-DO11 antibody (KJ126) was purchased from Caltag laboratories. IL-22, IL-17A, and IFN-γ concentrations were determined by ELISA on conditioned media from d5 of activation. Antibody pairs (coating, detection) were used to detect IFN-γ (AN-18, R4-6A2, Ebioscience), IL-17A (MAB721, BAF421, R&D Systems) and IL-22 (Ab-01, biotinylated Ab-03).

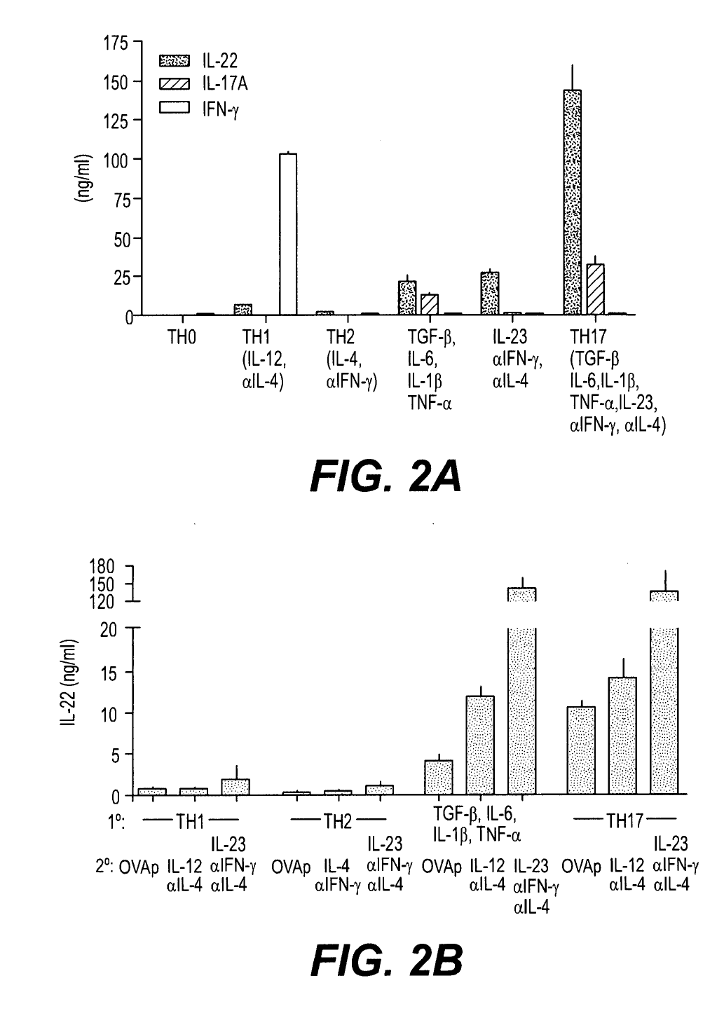

Naïve DO11 T cells activated with OVA₃₂₃₋₃₃₉(OVAp) only (Th0) produced minimal amounts of IL-22 (<100 pg/ml) (FIG. 2A). Although IL-22 expression was enhanced during Th1 (110 fold) and Th2 (40 fold) differentiation as compared to Th0, activation with IL-17 inducing conditions resulted in an even greater increase in IL-22 production. TGF-β, IL-6, IL-1β, and TNF-α enhanced IL-22 expression by 360 fold, whereas activation with IL-23, anti-IFN-γ, and anti-IL-4 increased IL-22 production by 460 fold. A combination of these conditions (Th17) yielded the greatest expression of IL-22, ˜2400 fold higher than Th0 and ˜22 fold higher than Th1. These data demonstrate that IL-22 protein is expressed most abundantly during Th17 differentiation.

Because some IL-22 was induced under Th1 and Th2 conditions during primary T cell activation, IL-22 production following a secondary stimulation of these cells was examined. Naïve DO11 cells were differentiated under Th1, Th2, or Th17 conditions or with TGF-β, IL-6, IL-1β, and TNF-α. On d7, cells were harvested, washed extensively, and rested overnight. 2×10⁵ DO11 T cells were restimulated with 4×10⁶ irradiated splenocytes, 5 ng/ml IL-2 (Sigma), and IL-12 and anti-IL-4, IL-4 and anti-IFN-γ, or IL-23, anti-IFN-γ, and anti-IL-4 were added as indicated. IL-22 concentrations were determined on day 5. Data shown are average±SD. (FIG. 2B)

Upon restimulation of these cells with OVAp and irradiated splenocytes, cells originally differentiated with TGF-β, IL-6, IL-1β, and TNF-α or with Th17 conditions produced at least 5 fold more IL-22. than Th1 or Th2 cells. (FIG. 2B) The continued differentiation of T cells along the Th17 lineage by restimulating with IL-23, anti-IFN-γ, and anti-IL-4 enhanced IL-22 production by at least 12 fold over restimulation of cells with OVAp alone or with IL-12 and anti-IL-4. In contrast, IL-22 production was not enhanced by restimulation of Th1 cells with IL-12, anti-IL-4 or of Th2 cells with IL-4, anti-IFN-γ. These results show that further differentiation towards Th1 or Th2 does not enhance IL-22 production. In addition, restimulation of Th1 and Th2 cells with IL-23, anti-IFN-γ, and anti-IL-4 did not enhance IL-22 production to that observed with Th17 cells activated under the same conditions. These data demonstrate that IL-23 is more potent than IL-12 in stimulating IL-22 expression and that Th17 cells are the major producers of IL-22.

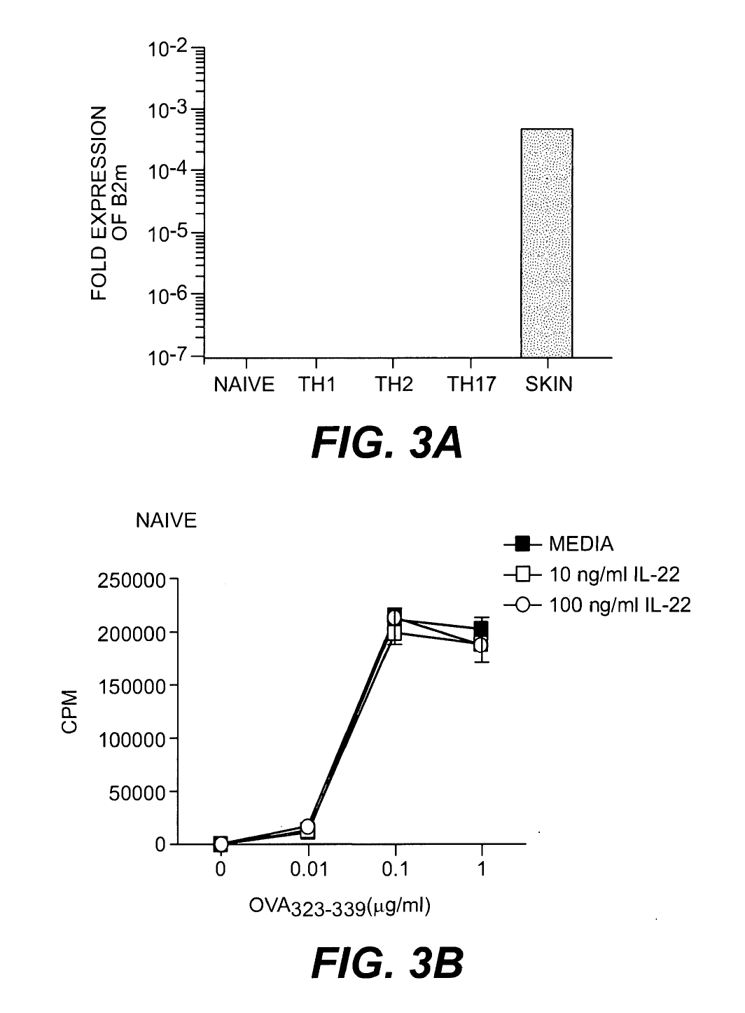

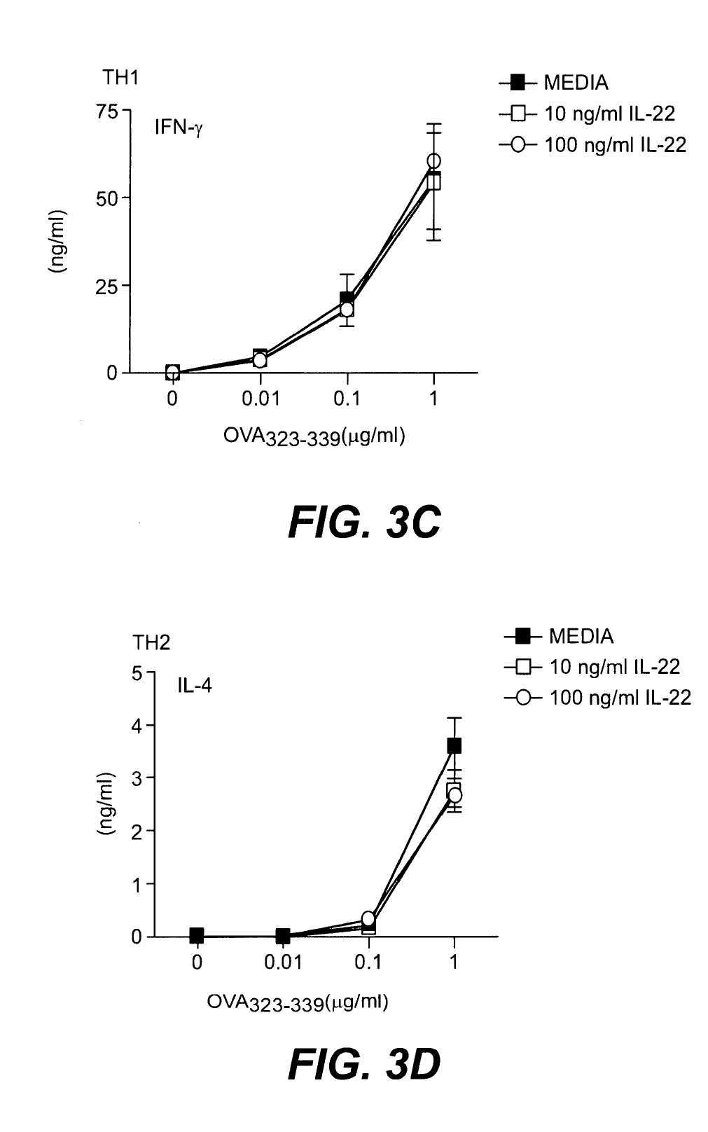

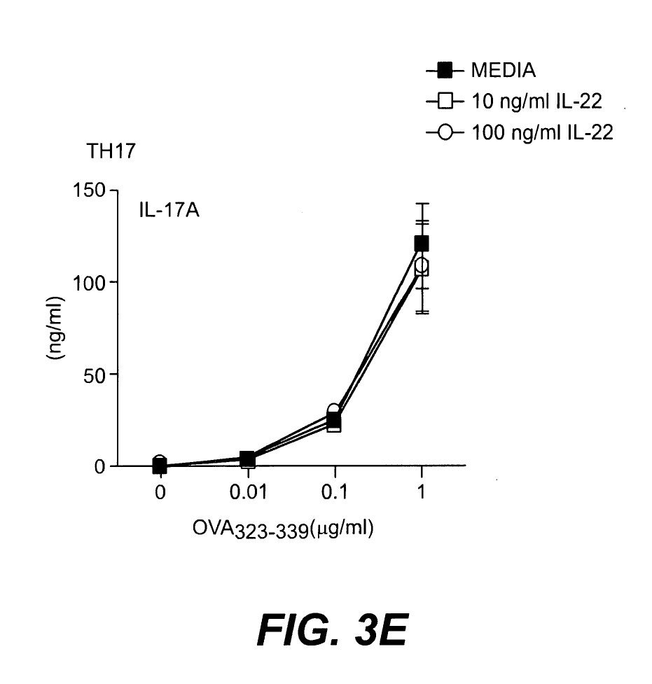

IL-22R1 transcript was not detected in any T cell population (FIG. 3A). The ability of IL-22 to modulate proliferation or IFN-γ, IL-4, and IL-17A production from naïve, Th1, Th2, and Th17 cells was also examined, but no changes were observed when T cells were treated with exogenous IL-22 (FIGS. 3B-3E). IL-17A or IL-17F also did not induce IL-22 expression from naïve, Th1, Th2, or Th17 cells. Thus, IL-22 and IL-17A/IL-17F do not directly modulate each other's expression by CD4 T cells.

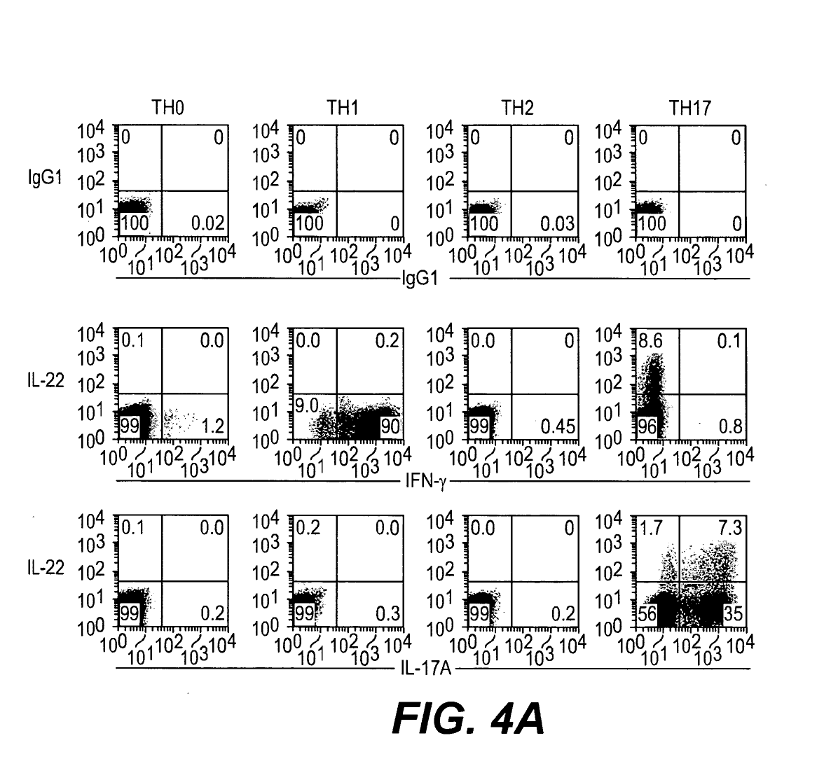

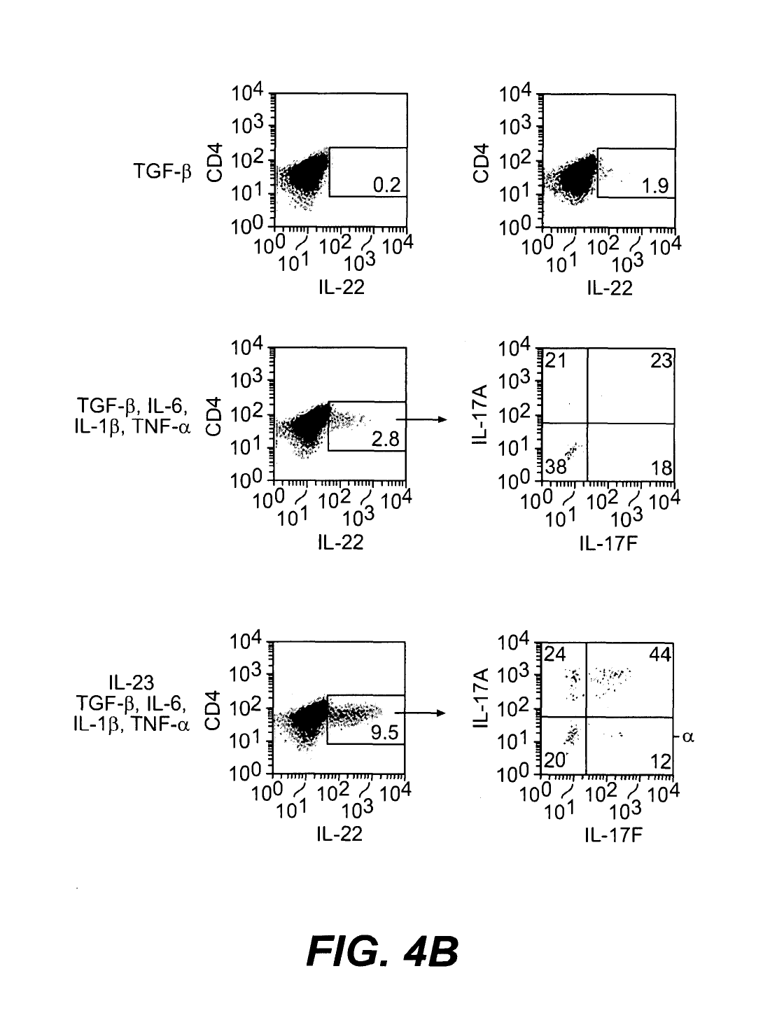

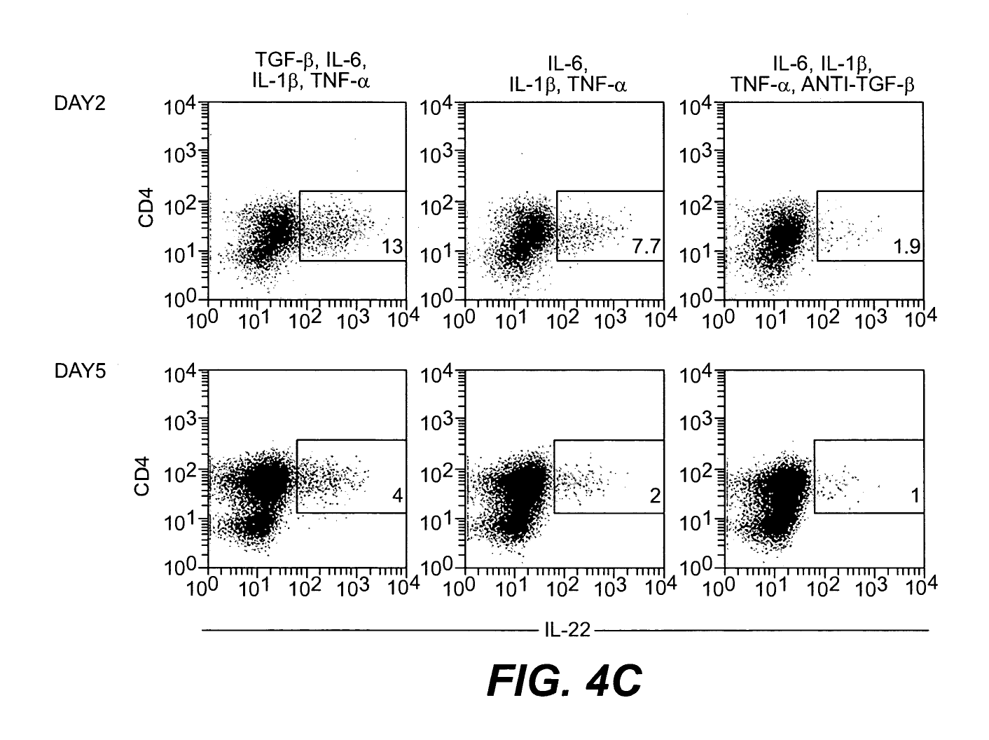

The induction of IL-22 during Th17 differentiation suggests that IL-22 and IL-17 can be co-expressed by the same T cell. To examine this, intracellular cytokine staining was performed on T cells activated under various conditions. Intracellular cytokine staining for IFN-γ, IL-17A, and IL-22 was performed on cells from FIG. 2A on d5 of activation. Cells were restimulated with 50 ng/ml PMA (Sigma), 1 μg/ml ionomycin (Sigma), and GolgiPlug (Pharmingen) for 6 hours. Cells were first stained for surface antigens and then treated with Cytofix/Cytoperm (Pharmingen) according to manufacturer's directions. Intracellular cytokine staining was performed using antibodies to IFN-γ, IL-22, IL-17A, and IL-17F. Anti-IL-22 (-02) was labeled with Alexa 647 (Molecular Probes) and anti-IL-17F (15-1) was labeled with FITC (Pierce Biotechnologies) according to manufacturer's directions. All plots are gated on KJ126⁺CD4⁺ cells and positive percentages shown. Th0, Th1, and Th2 activated cells had minimal expansion of IL-22 producing cells (≦0.2%) (FIG. 4A). Activation under Th17 conditions generated a substantial population of IL-22 expressing cells (8.7%), with 81% of IL-22⁺ cells expressing IL-17A and only 1% expressing IFN-γ.

The roles of individual cytokines under Th17 differentiation conditions were further examined. Naïve DO11 T cells were activated with 1 μg/ml OVAp, irradiated splenocytes, and the indicated cytokines. Intracellular cytokine staining for IL-17A, IL-17F, and IL-22 was performed on d5 of activation. Data are representative of 3 experiments. Only 0.2% of cells activated with exogenous TGF-β expressed IL-22 (FIG. 4B). Activation with IL-6, IL-1β, and TNF-α enhanced IL-22⁺ cells (1.9%). Addition of exogenous TGF-β to IL-6, IL-1β, and TNF-α further increased IL-22⁺ cells (2.8%), with 62% of IL-22⁺ cells expressing IL-17A or IL-17F. Activation with IL-23, along with TGF-β, IL-6, IL-1β, and TNF-α, led to an ˜3 fold increase in IL-22⁺ cells (9.5%) (FIG. 4B). Eighty percent of IL-22⁺ cells produced either IL-17A or IL-17F, with the majority of cells expressing both IL-17A or IL-17F (44%) (FIG. 4B). The addition of a neutralizing antibody to TGF-β indicated that exogenous TGF-β is important for optimal expression of IL-22 induced by IL-6, IL-1β and TNF-α (FIG. 4C). In summary, these data demonstrate that IL-22 protein is produced in greater amounts by Th17 cells and that IL-22 is co-expressed with both IL-17A and IL-17F during Th17 differentiation.

### Example 3

IL-23 enhances the expansion of IL-22 producing cells during Th17 differentiation.

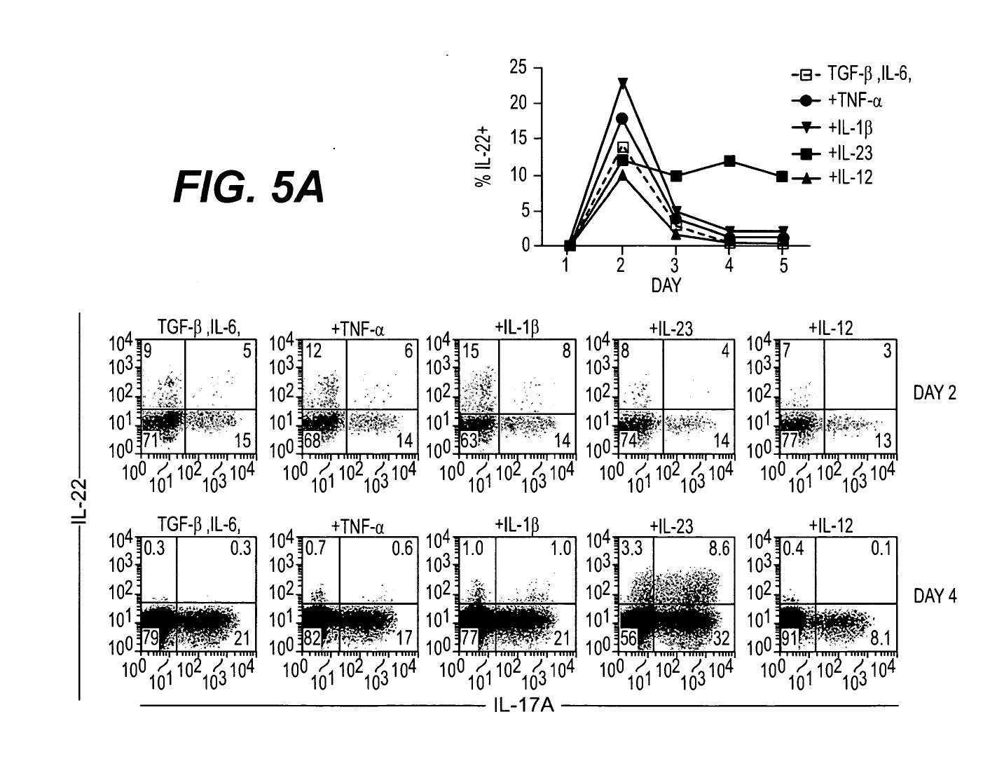

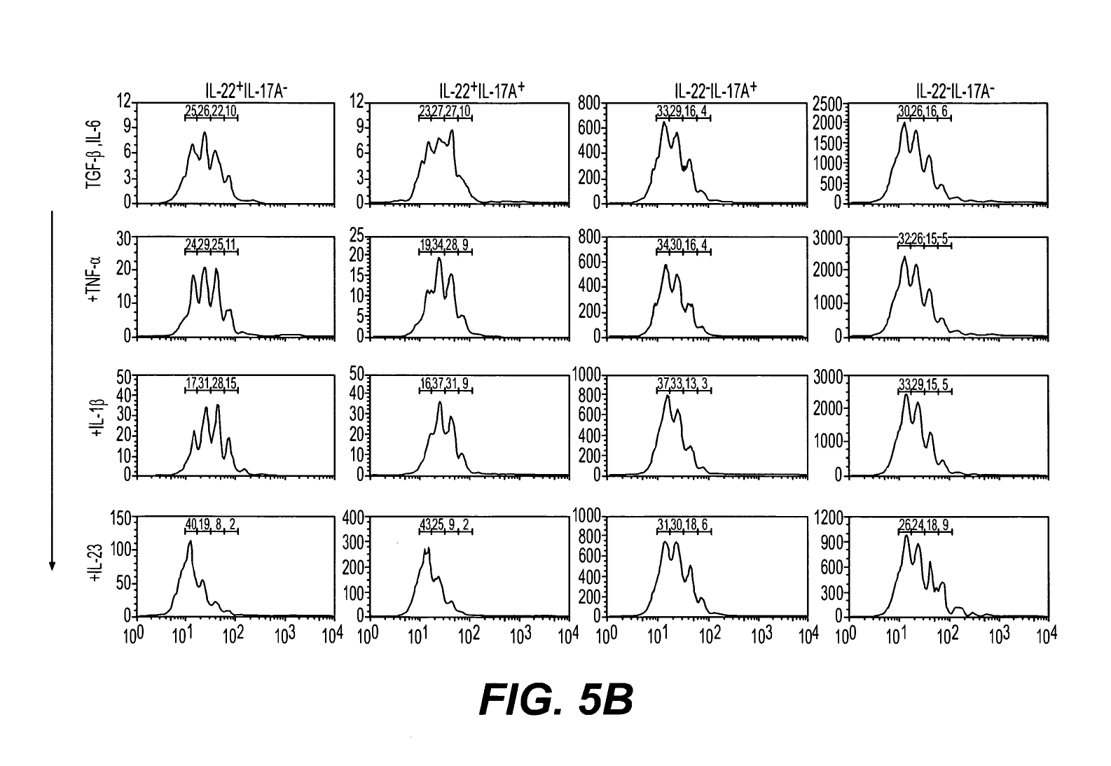

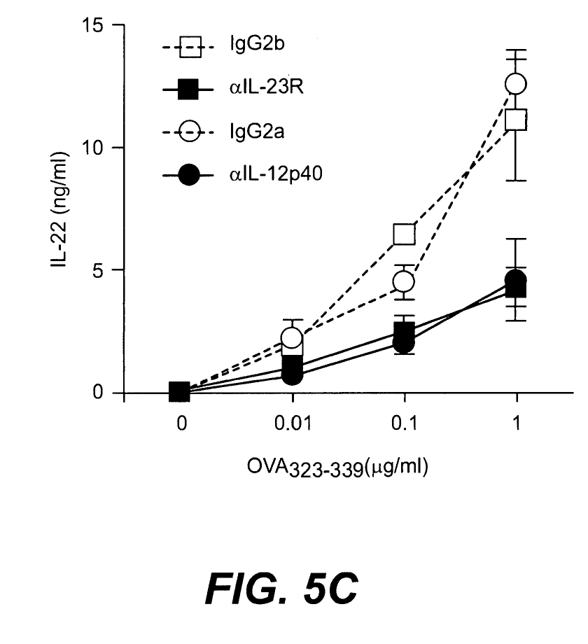

To further examine how IL-23 enhances IL-22 expression during Th17 differentiation, naïve DO11 T cells labeled with CFSE (Molecular Probes) were differentiated with 1 μg/ml OVAp irradiated splenocytes, TGF-β, and IL-6. TNF-α, IL-1β, IL-23 or IL-12 was added to some cultures. The expression of IL-22 was analyzed from d1 to d5 of culture. Intracellular cytokine staining for IL-22 and IL-17A was performed on d1 through d5. The percentages of IL-22⁺ cells on d1-d5 were determined. FIG. 5A shows the percentage of cells expressing IL-22 plotted as a function of time and representative flow cytometry plots from d2 and d4. Cells activated with only TGF-β and IL-6 peaked in IL-22 (15%) expression on d2 and decreased substantially by d3. Neither TNF-α, IL-1β, nor IL-12 addition prevented the decrease in expression of IL-22 observed after d2. In contrast, cells activated with IL-23, TGF-β, and IL-6 expressed at least 5 fold more IL-22 on day 4.

To examine if IL-23 was inducing the expansion of IL-22 producing cells, we analyzed the CFSE dilution profiles of cells expressing IL-22 and/or IL-17A on d4 (FIG. 5B). No differences in CFSE were observed between IL-22⁻IL-17A⁺ and IL-22⁻ IL-17A⁻ cells activated with TGF-β and IL-6 alone, or when supplemented with IL-1β, TNF-α, or IL-23. This suggests that there is no correlation between IL-17A expression and proliferation. CFSE profiles of IL-22⁺IL-17A⁻ and IL-22⁺IL-17A⁺ cells activated with TGF-β and IL-6 indicated that these cells had proliferated less than IL-22⁻IL-17A⁻ and IL-22⁻IL-17A⁺ cells. Similar findings were observed in cultures supplemented with IL-1β, TNF-α or IL-12. In contrast, IL-23 in the context of TGF-β and IL-6 enhanced the proliferation and expansion of IL-22⁻IL-17A⁻ and IL-22⁺IL-17A⁺ cells. These findings demonstrate that IL-23 drives the expansion of IL-22 producing cells in the Th17 lineage.

To examine if endogenous IL-23 is necessary for optimal IL-22 expression, naïve DO11 T cells were activated with LPS treated dendritic cells (“DCs”), OVAp, and neutralizing antibodies to IL-23R or to IL-12p40. To generate DCs, bone marrow cells were cultured with 10 ng/ml GM-CSF and 1 ng/ml IL-4 for 7 days. After purification by CD11c positive selection (Miltenyi Biotec), DCs were matured for 24 hours with 1 μg/ml LPS (E. Coli Serotype 0111-B4, Sigma). DCs were then washed, and 1×10⁴ DCs were cultured with 2×10⁴ purified naïve DO11 T cells, OVAp, and 10 μg/ml anti-IL-12p40, anti-IL-23R, or relevant isotype controls.

Anti-IL-12p40 (C17.8) and anti-IL-23R (258010) were obtained from R&D Systems. IL-22 concentrations were determined on d5 of culture. Data are representative of at least 2 experiments. Neutralization of IL-23R reduced IL-22 production by 62% (at 1 μg/mL OVAp) as compared to isotype control (FIG. 5C). A similar reduction of IL-22 expression was observed with anti-IL-12p40 (64%), suggesting that IL-23, and not IL-12, is responsible for the majority of IL-22 production. Taken together, these data demonstrate that IL-23 induces optimal expansion of IL-22 producing cells.

### Example 4

Expression of mouse IL-22 requires IL-6 and IL-23.

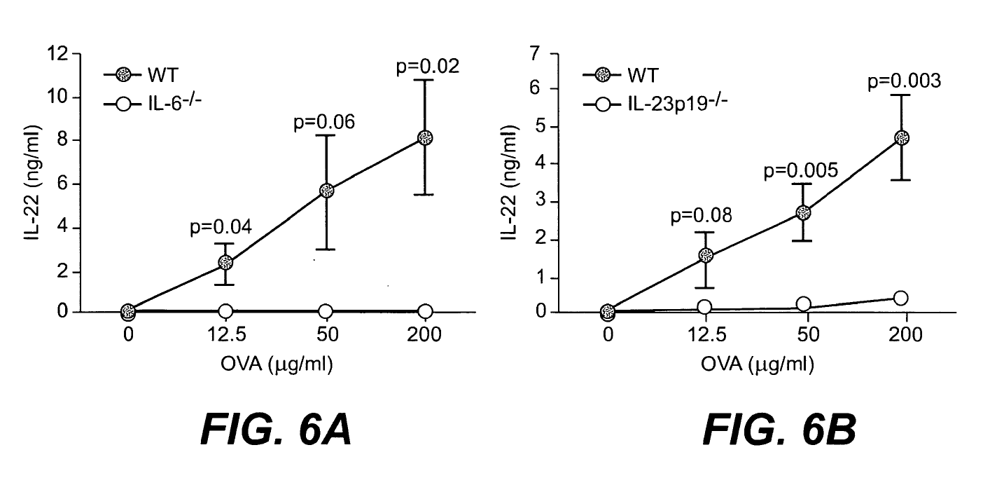

IL-23 can induce expression of IL-22 from mouse T cells in vitro. To examine how IL-23 affects IL-22 expression in vivo, C57BL/6 IL-23p16 deficient mice (7 mice per group) were immunized with 100 μg of OVA emulsified in CFA. The C57BL/6 IL-23p19 deficient mice were generated as previously described (Thakker, P. et al., J. Immunol. (2007) 178:2589-2598). IL-6 deficient mice (B6; 129S2-II6tm1Kopf/J; Jackson Laboratories, five mice per group) were also immunized to examine how IL-6 affects IL-22 expression in vivo. Ten days after immunization, draining inguinal lymph nodes (“LN”) were harvested and restimulated in the presence of OVA ex vivo. IL-22 concentrations were determined on day four of ex vivo restimulation. Mice deficient in either IL-23 or IL-6 produced significantly less IL-22 as compared to their respective WT controls (FIG. 6; data representative of at least two experiments). Thus, both IL-23 and IL-6 are required for optimal differentiation of IL-22 expressing cells in vivo.

### Example 5

IL-22 does not act on naïve or differentiated T cells.

The functional receptor for IL-22 is composed of a heterodimer complex between IL-22R1 and IL-10R2. While IL-10R2 is expressed ubiquitously in all tissues, IL-22R1 is restricted primarily to non-lymphoid tissues and cells. Although expression of IL-22R1 is not detected on naïve or 3-day activated human peripheral blood lymphocytes, it is not known if differentiated murine Th1, Th2, or Th17 cells can express IL-22R1. To examine this, quantitative PCR for IL-22R1 was performed in naïve as well as differentiated DO11 cells. Expression of IL-22R1 was not detected in naïve, Th1, Th2, or Th17 T cells. In contrast, IL-22R1 was positively detected in skin. While IL-22R1 is not expressed on T cells, it is possible that IL-22 could signal through a yet unidentified receptor. To examine functionally if IL-22 can act on naïve or differentiated T cells, naïve, Th1, Th2, and Th17 T cells were activated in the presence of IL-22. No consistent effects on proliferation and cytokine production (IFN-γ, IL-4, IL-17A) by naïve or differentiated Th1, Th2, or Th17 cells were observed with addition of exogenous IL-22 up to 100 ng/ml. These data indicate that IL-22 does not act on, naïve or differentiated T cells.

### Example 6

IL-22 is co-expressed with IL-17A and IL-17F in vivo.

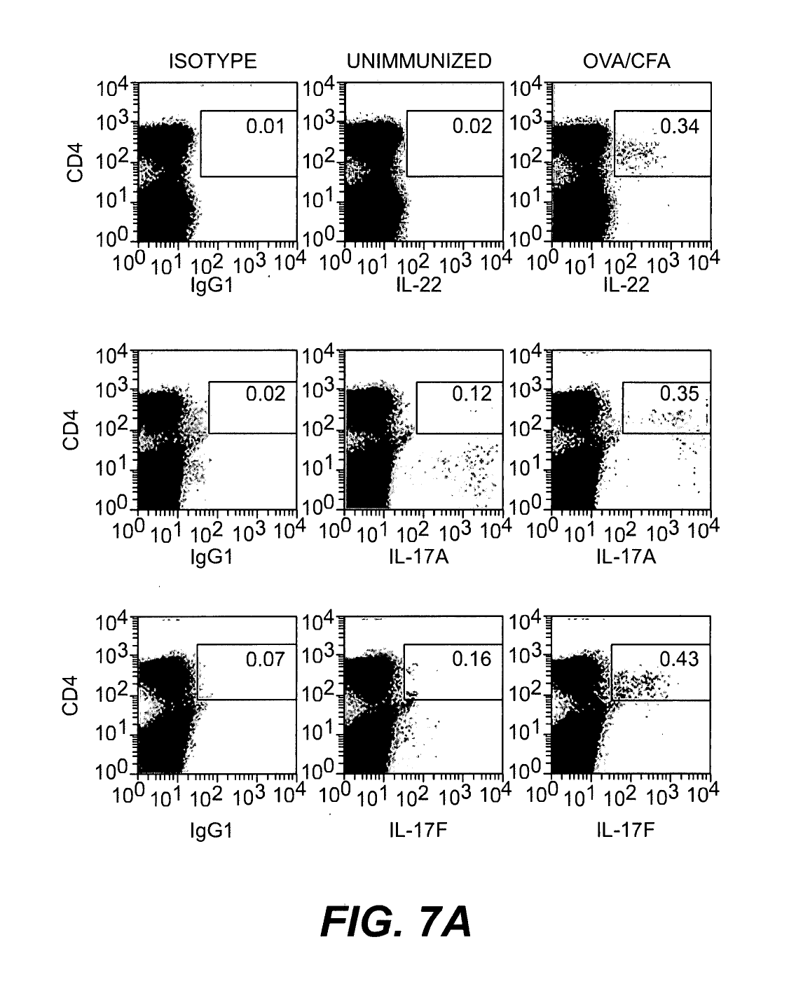

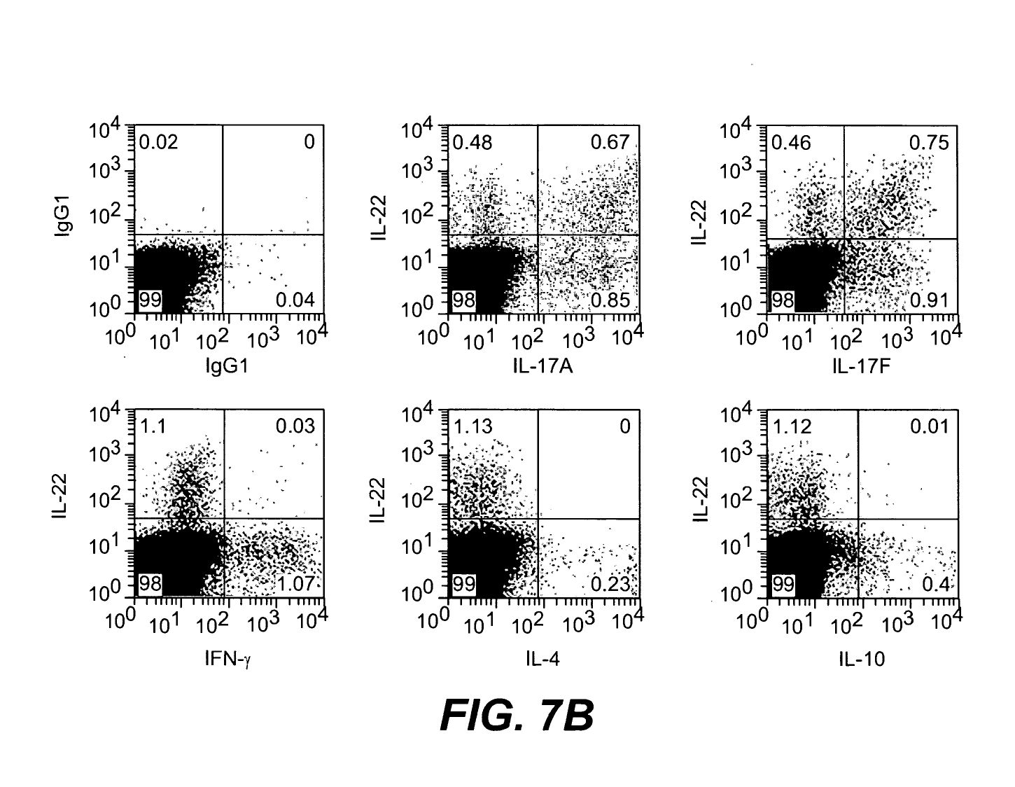

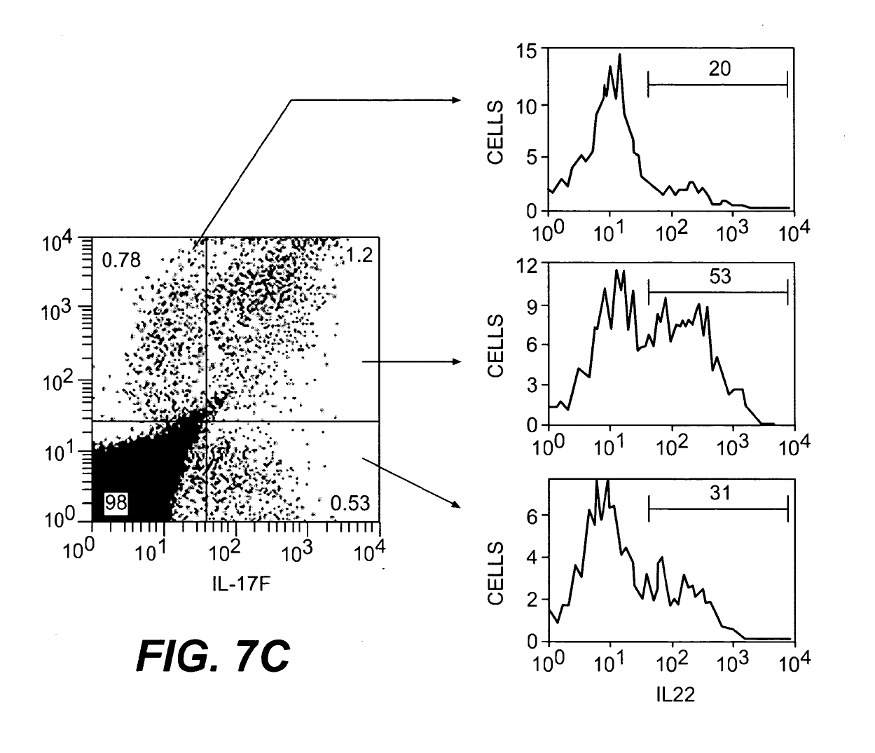

The in vitro data demonstrate that IL-22 is co-expressed with IL-17A and IL-17F. To examine if this population exists in vivo, C57BL/6 mice were immunized sub-cutaneously with 100 μg OVA (Sigma) emulsified in CFA (Sigma). Seven days later, intracellular cytokine staining was performed on draining LN directly ex vivo. Immunization with OVA/CFA increased the expansion of IL-22⁺ (0.34%), IL-17A⁺ (0.35%) and IL-17F⁺ (0.43%) cells as compared to unimmunized mice (FIG. 7A). IL-22 was co-expressed with IL-17A (44% of IL-17A⁺ cells were IL-22⁺) and IL-17F (45% of IL-17F⁺ cells were IL-22⁺) but not with IFN-γ, IL-4, or IL-10 (FIG. 7B). When the expression between IL-17A and IL-17F was compared, considerable, but not complete, co-expression was detected between the two cytokines (FIG. 7C). IL-17A⁺IL-17F⁺ cells comprised 60% of IL-17A⁺ and 70% of IL-17F⁺ cells. The results demonstrate heterogeneity of IL-17A and IL-17F expression within Th17 cells. No co-expression of IL-17F with IFN-γ, IL-4, or IL-10 was observed. IL-22 expression in IL-17A and/or IL-17F producing cells was also measured and the highest IL-22 expression was found to be in IL-17A⁺IL-17F⁺ cells (53.1%) (FIG. 7C). The expression of IL-17A and IL-17F in IL-22⁺ cells was analyzed as well (FIG. 7D). Seventy percent of IL-22⁺ cells expressed either IL-17A or IL-17F, with 45% of IL-22⁺ cells expressing both.

The in vivo expression profiles among IL-17A, IL-17F, and IL-22 are similar to the expression profiles generated in vitro with TGF-β, IL-6, IL-1β, TNF-α, and IL-23 (See FIG. 4B), suggesting that this in vitro condition is sufficient to replicate in vivo Th17 differentiation. Similar expression patterns for IL-22, IL-17A, and IL-17F were also observed on d4 and d10 after immunization. These data demonstrate that IL-22 is not co-expressed with IFN-γ, IL-4, and IL-10 in vivo, but rather with IL-17A and IL-17F.

To examine if IL-23 stimulates IL-22 production from in vivo primed T cells, LN cells were restimulated with 200 μg/ml OVA, OVA and IL-12, OVA and IL-23, or with medium alone. IL-22 and IL-17A concentrations were examined on d4 of restimulation. The ELISA data shown in FIG. 7E are average±SD and are representative of three independent experiments. Addition of IL-23 enhanced the production of IL-22 by 7 fold compared to OVA alone while exogenous IL-12 had no effect. These data further support that IL-23, rather than IL-12, is the stimuli for enhancing IL-22 production.

### Example 7

IL-22 is expressed by human Th17 cells and, to a lesser extent, human Th1 cells.

To investigate if IL-22 is also expressed by human Th17 cells, CD4⁺ T cells from six separate donors were activated with allogeneic CD4-depleted peripheral blood lymphocytes (“PBLs”) in a mixed lymphocyte reaction (MLR) under various stimulation conditions. Human CD4⁺ T cells were purified from peripheral blood of donors by Rosette Sep (Stem cell technologies). In a 48 well plate, 7.5×10⁵ human T cells were cultured with 7.5×10⁵ irradiated (3300 rads) CD4-depleted PBLs from a separate donor. The indicated cytokines and antibodies were added at the following concentrations: 20 ng/ml IL-6, 10 ng/ml IL-1β, 10 ng/ml TNF-α, 1 ng/ml TGF-β, 10 μg/ml anti-IL-4 (MP4-25D2, Pharmingen), 10 μg/ml anti-IFN-γ (NIB412, Pharmingen) and 10 μg/ml anti-TGF-β (1D11, R&D Systems).

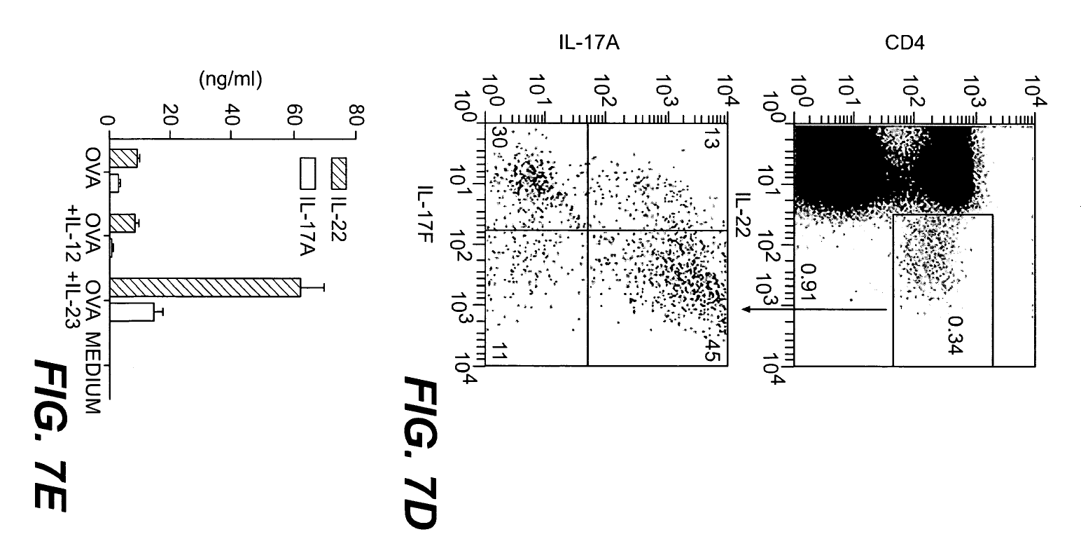

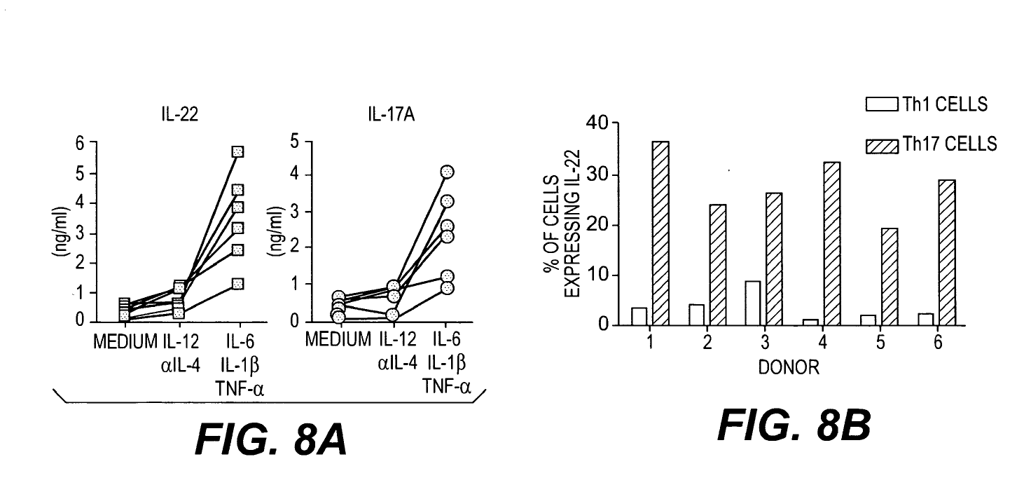

On day 7 of activation, the conditioned medium was harvested and the human IL-22 present was quantified by coating plates with 2.5 μg/ml of anti-human IL-22 antibody (Ab-04) and detecting with 1 μg/ml of anti-human IL-22 antibody (354A08), followed by biotinylated anti-human IgG (Pharmingen 341620) and streptavidin HRP. Human IL-17A concentrations in the conditioned medium were determined by ELISA coating with 4 μg/ml anti-human IL-17A (MAB317, R&D Systems) and detecting with 75 ng/ml biotinylated anti-human IL-17A (BAF317, R&D Systems) and streptavidin HRP. CD4⁺ T cells from six individual donors were examined. In the absence of any exogenous cytokine, IL-22 was produced in low amounts (<600 pg/ml) (FIG. 8A, each line represents a distinct donor). Activation with a Th1 condition using IL-12 and neutralizing antibody to IL-4 enhanced the expression of IL-22 by an average of 2.5 fold. Activation with a Th17 condition using IL-6, IL-1β, and TNF-α resulted in greater expression of IL-22, increasing production by an average of 17 fold. IL-17A expression was enhanced to a greater extent under the Th17 condition (9.5 fold) than under the Th1 condition (1.4 fold) (FIG. 8A). These data indicate that, as for mouse T cells, activation of human CD4 T cells with IL-6, IL-1β, and TNF-α greatly increased production of both IL-22 and IL-17A.

The expression of IL-22 was also examined by intracellular cytokine staining to determine what kind of CD4 T cells are producing IL-22 in our MLR system. Cells activated under a Th1 differentiation condition (IL-12, anti-IL-4) or a Th17 condition (IL-6, IL-1β, TNF-α) were restimulated with 50 ng/ml PMA, 1 μg/ml ionomycin, and GolgiPlug (Pharmingen) for 5 hours, fixed, and permeabilized with Cytofix/Cytoperm (Pharmingen). Intracellular co-staining of CD4⁺ T cells for IL-22, IL-17A, and IFN-γ was performed using anti-IL-22 PE (R&D systems), anti-IFN-γ FITC (Pharmingen), anti-CD4 PerCp-Cy5.5 (Pharmingen), and anti-IL-17A 647 (R&D Systems). Th1 cells were defined by the expression of IFN-γ and Th17 cells were defined by their expression of IL-17A. The percentage of Th1 or Th17 cells expressing IL-22 were calculated for each of the six donors examined. Data are representative of at least two experiments. Although some IL-22 expression was detected in Th1 cells, IL-22 expression was consistently higher in Th17 cells than in Th1 cells in all six donors (FIG. 8B). These data indicate that IL-22 is produced by human Th17 cells and, to a lesser extent, by human Th1 cells.

### Example 8

TGF-β inhibits expression of IL-22 from human T cells.

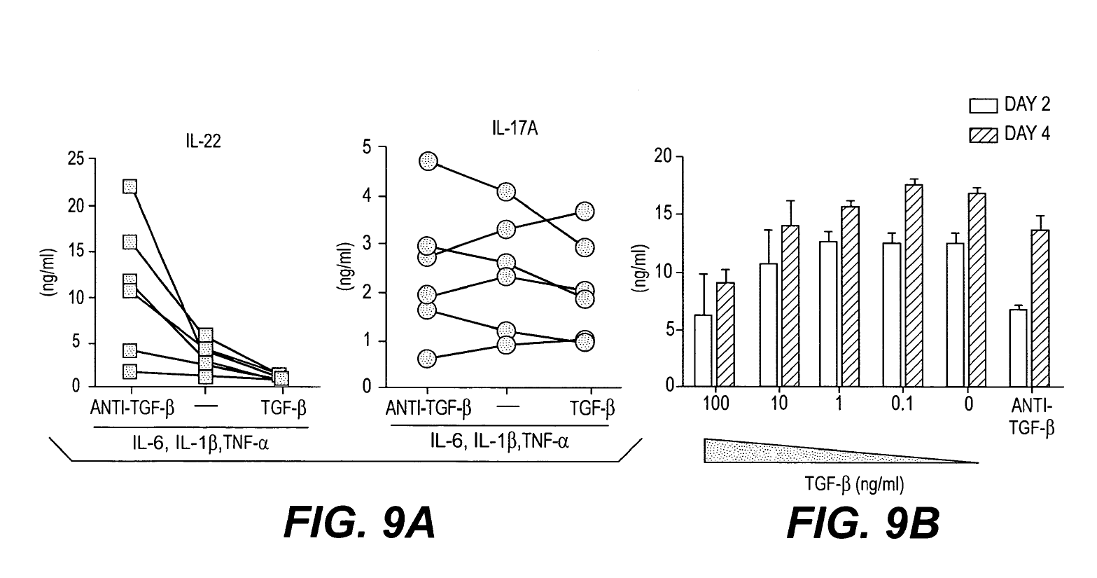

Exogenous TGF-β and IL-6 support the differentiation of Th17 cells in mice, with IL-1β and TNF-α further augmenting the response (Veldhoen, M. et al., Immunity (2006) 24:179-89; Mangan, P. R. et al., Nature (2006) 441:231-34; Bettelli, E. et al., Nature (2006) 441:235-38). IL-22 expression from human cord blood derived naïve CD4 T cells activated with anti-CD3, anti-CD28, and IL-6 was reduced by exogenous TGF-β, indicating that TGF-β is not only dispensible for human IL-22 expression, but acts to inhibit it (Zheng, Y. et al., Nature (2007) 445:648-651). To examine the role of TGF-β in a MLR where APCs are present, CD4⁺ T cells from six donors were activated with IL-6, IL-β, and TNF-α alone, or further supplemented with either exogenous TGF-β cytokine (Sigma Aldrich) or a neutralizing antibody to human TGF-β (1D11, R&D Systems). IL-22 and IL-17A concentrations in day 7 conditioned media from MLR were determined. As TGF-β can be made by lymphocytes, addition of an anti-TGF-β antibody is needed to prevent endogenous TGF-β signaling. Neutralization of TGF-β in the context of IL-6, IL-1β, and TNF-α enhanced IL-22 expression by an average of 3.0 fold, indicating that TGF-β inhibits production of IL-22 by human T cells (FIG. 9A, each line represents a distinct donor). Consistent with this observation, exogenous TGF-β added to IL-6, IL-1β, and TNF-α reduced IL-22 production by an average of 4.4 fold. The role of TGF-β on IL-17A expression was also examined in our human MLR system. Adding either a neutralizing antibody to TGF-β or the TGF-β cytokine had no consistent effects (<1.2 fold average change) on IL-17A production as induced by IL-6, IL-1β, and TNF-α. Therefore, these data demonstrate that TGF-β inhibits IL-22 expression by PBL-derived CD4⁺ human T cells, but that it has no substantial effect on IL-17A expression.

The role of TGF-β in regulating mouse IL-22 expression was also examined. Naïve CD62L⁺ DO11 T cells were activated with IL-6 and with either TGF-β cytokine or a neutralizing antibody to TGF-β. IL-22 expression was examined by ELISA on day two and day four of activation. Although IL-22 expression does not require the presence of exogenous TGF-β, neutralization of endogenous TGF-β with an antibody consistently reduced expression of IL-22 (˜1.8 fold) on day 2 of activation, indicating that the presence of endogenous TGF-β does contribute to enhancing IL-22 production (FIG. 9B) in murine T cells. By day four, neutralization of TGF-β did not have as large an effect on IL-22 expression, suggesting that TGF-β has its greatest effect on enhancing IL-22 expression during the initial activation. Interestingly, addition of high amounts of exogenous TGF-β (>=10 ng/ml) inhibited IL-22 expression on both day two and day four of activation. Taken together, these data indicate that in the presence of IL-6, endogenous TGF-β signaling enhances mouse IL-22 production during initial stages of activation whereas addition of large amounts of exogenous TGF-β actually inhibits IL-22 expression.

### Example 9

IL-22 administration via adenoviral vectors effects an acute phase response in mice.

IL-22 expression by both mouse and human T cells can be induced by IL-6, IL-1β, and TNF-α. These pro-inflammatory cytokines are known to induce an acute phase response. An acute phase response is a collection of biochemical, physiologic, and behavioral changes indicative of an inflammatory condition. The modulation of specific proteins known as acute phase reactants is a biochemical hallmark of an acute phase response and of inflammation. Treatment of hepatocytes with IL-22 in vitro and administration of IL-22 in vivo can rapidly induce the expression of serum amyloid A (SAA), a major acute phase reactant (Dumoutier, L. et al., Proc. Nat'l Acad. Sci. U.S.A. (2000) 97:10144-49; Wolk, K. et al., Immunity (2004) 21:241-54).

To study the role of IL-22 in a more chronic setting, IL-22 was ectopically expressed in C57BL/6 mice using a replication-defective adenovirus. Expression of acute phase reactants was examined up to two weeks after administration. SAA expression was significantly enhanced as compared to GFP-expressing adenovirus starting on day three and remained significantly increased up to 14 days. later (data not shown). Fibrinogen, another acute phase reactant, was also significantly enhanced in mice administered the IL-22 expressing adenovirus, starting as early as day one and remaining significant up to seven days later (data not shown). Whereas some proteins are induced during an acute phase response, other proteins, such as albumin, are decreased during inflammation. Mice treated with IL-22 expressing adenovirus exhibited decreased expression of albumin as compared to the GFP expressing control (data not shown). These data demonstrate that exposure to IL-22 for two weeks using an adenovirus for ectopic expression results in the modulation of several proteins indicative of an acute phase response.

The effects of IL-22 adenoviral administration on blood cells were also investigated. Mice treated with the IL-22 expressing adenovirus resulted in a significant increase in serum platelet seven days (1.5 fold) and 14 days (2.0 fold) after viral inoculation relative to the GFP adenoviral control (data not shown). Concomitant with this increase in platelet number, a mild anemia indicated by a modest, but statistically significant decrease in red blood cells was observed. Similarly significant decreases were also detected in both the serum hematocrit and hemoglobin (data not shown). A trend of increased numbers of segmented neutrophils in the blood was also found, although the increase was not always significant. Taken together, the biochemical and hematological changes we observed in mice treated with an IL-22 expressing adenovirus indicate that IL-22 induces an acute phase response (APR) in vivo.

### Example 10

IL-22 protein can directly enhance SAA in the absence of IL-6.

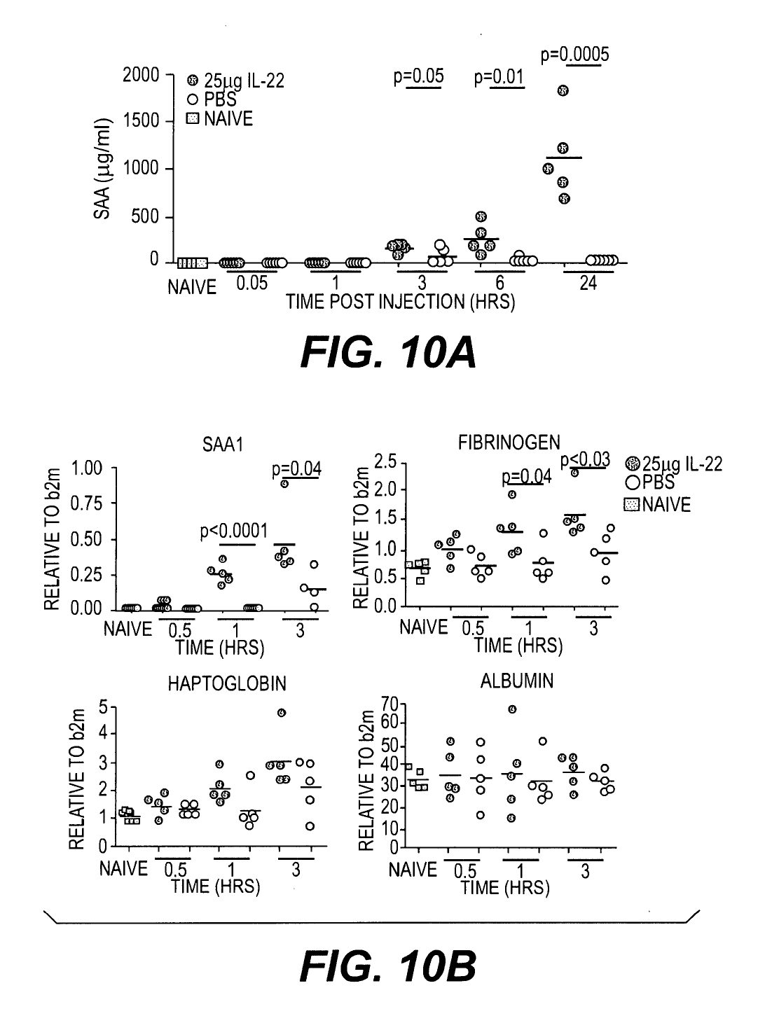

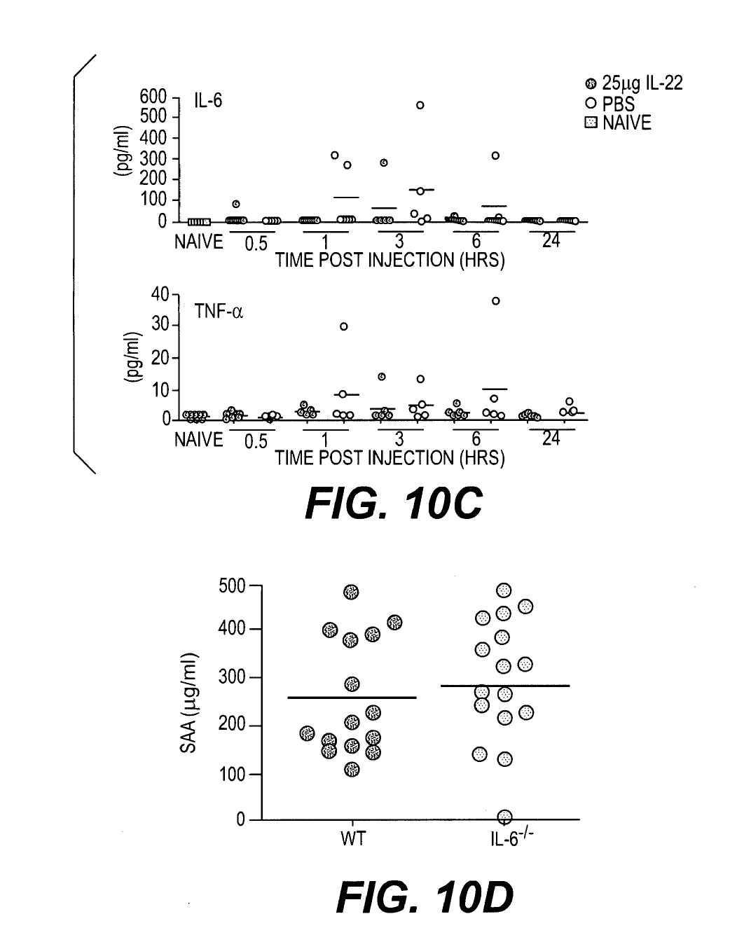

Our data using adenoviral constructs demonstrated that IL-22 is capable of modulating parameters indicative of an acute phase response in vivo. However, it was possible that IL-22 was acting with other factors as a result of infection with adenovirus. To directly examine the role of IL-22, IL-22 protein was administered to mice by intraperitoneal injection and the serum was examined at several timepoints for changes in acute phase reactants. Mice were administered 25 μg of IL-22 protein or PBS via intraperitoneal injection. Mouse IL-22 was generated using methods previously described (Li, J., et al., Int. Immunopharmacol. (2004) 4:693-708). Blood and liver were harvested at 0.5, 1, 3, 6, and 24 hours and serum prepared. SAA was quantified using a SAA-specific ELISA (Invitrogen). Administration of IL-22 protein was sufficient to significantly enhance expression of SAA protein in the serum starting at 3 hours after administration and up to 24 hours (FIG. 10A).

Livers from the mice administered IL-22 or PBS were also snap frozen and then processed for RNA using the Ribopure RNA isolation kit (Ambion). Quantitative PCR was performed using Taqman (Applied Biosystems) and pre-qualified primer/probes (Applied Biosystems) for SAA1, fibrinogen, haptoglobin, and albumin. The relative amounts of each gene, as normalized to β2 microglobulin, were then calculated. SAA transcript expression in the liver was increased by 0.5 hour after administration and was significantly increased at one hour and three hours (FIG. 10B). In addition to SAA, IL-22 was observed to significantly enhance fibrinogen transcripts in the liver within 1 hour after injection (FIG. 10B). Haptoglobin and albumin transcripts were not statistically changed at up to 3 hours after injection (FIG. 10B). Thus, IL-22 can begin to effect changes of an acute phase response within 1 hour after intraperitoneal administration.

Although IL-22 injection induced SAA, it was possible that IL-22 was acting indirectly by inducing other cytokines such as IL-6 and TNF-α that then directly enhanced SAA expression. To examine if IL-22 induces IL-6 and TNF-α in vivo, serum IL-6 and TNF-α expression was examined after IL-22 administration. Concentrations of IL-6 and TNF-α were determined using the Inflammation CBA kit (Pharmingen). No significant changes were observed up to 24 hours post administration (FIG. 10C). As it was possible that the amounts of IL-6 or TNF-α produced were too low to be detected, IL-22 protein was also directly administered to IL-6 deficient mice. C57BL/6 and C57BL/6 IL-6^{-/-} mice were administered 25 μg of IL-22 via intraperitoneal injection. Mice were bled at six hours after injection and SAA quantified from the serum. Fifteen mice were examined per group, and the data shown are representative of at least two experiments. The absence of IL-6 had no effects on IL-22-induced SAA production (FIG. 10D) in IL-6 deficient mice. Taken together, these data support earlier studies showing that IL-22 modulates parameters indicative of an acute phase response. These data further indicate that IL-22 can regulate SAA, a major acute phase reactant, in the absence of IL-6 signaling.

### Example 11

IL-22 induces neutrophil mobilization in the blood.

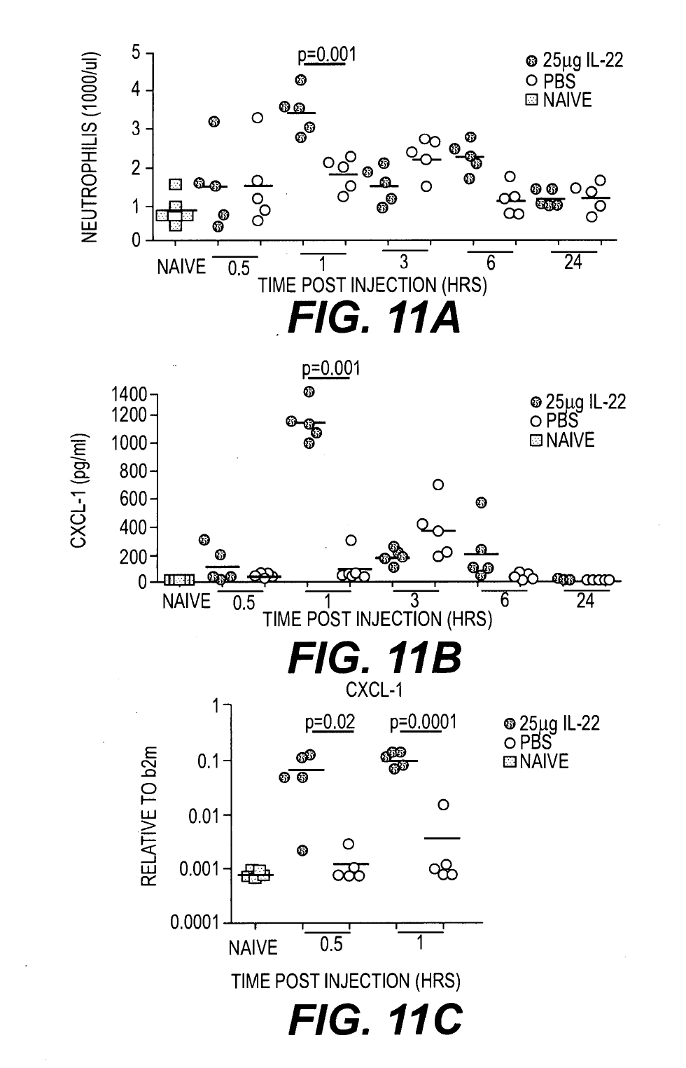

The hematological changes that result from IL-22 protein administration were also examined. Mice were administered 25 μg of IL-22 protein or PBS via intraperitoneal injection. Blood was collected at several timepoints after administration and neutrophil numbers quantified using a Cell-Dyn hematology analyzer (Abbott Diagnostics). IL-22 induced a significant, two fold increase in neutrophil counts in the blood one hour after administration (FIG. 11A). This increase was transient as no statistically significant changes were observed after one hour. Expression of several neutrophil chemoattractants was also examined. A significant increase in CXCL1 (13 fold) was detected in the serum at one hour after administration (FIG. 11B). CXCL1 was quantified using a CXCL1-specific ELISA (R&D Systems) following the manufacturer's directions. Quantitative PCR revealed that CXCL1 transcripts in the liver were also significantly enhanced starting at 0.5 hour after injection (FIG. 11C). Data are representative of at least three experiments. No increases in CXCL2, CXCL5, or G-CSF were observed in the serum at any timepoint. These data demonstrate that IL-22 can induce neutrophil mobilization and the expression of the neutrophil chemoattractant, CXCL1, possibly from the liver.

### Example 12

IL-22, IL-17A, and IL-17F cooperatively induce anti-microbial peptides.

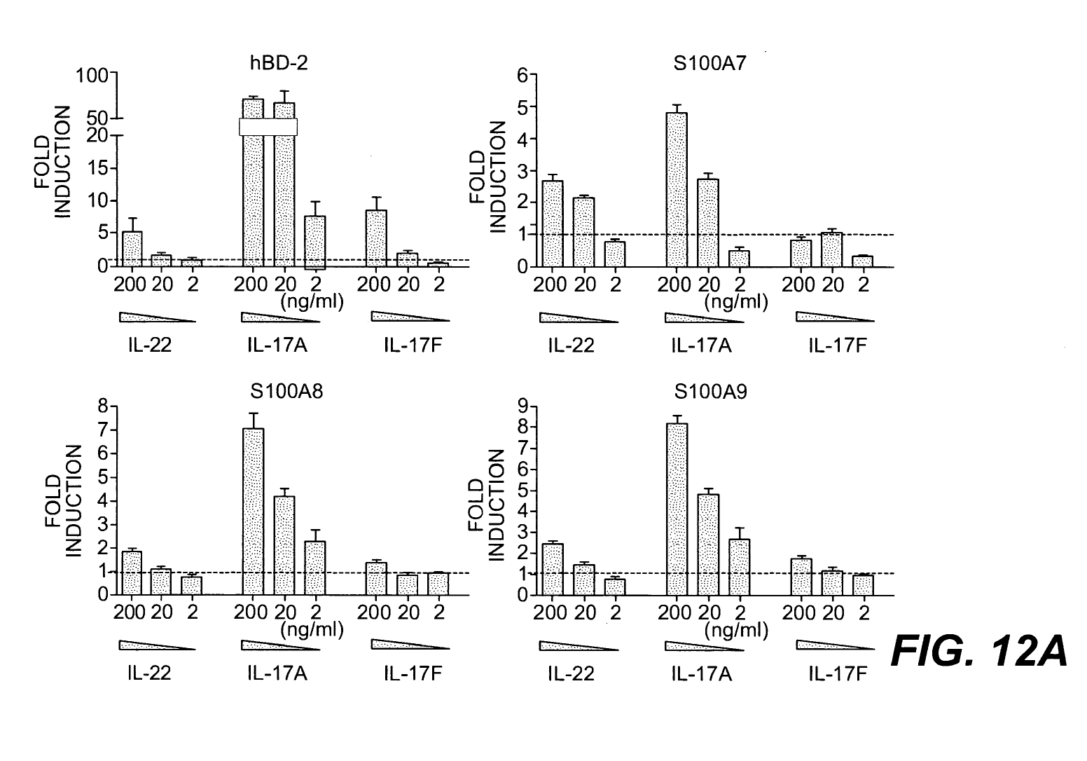

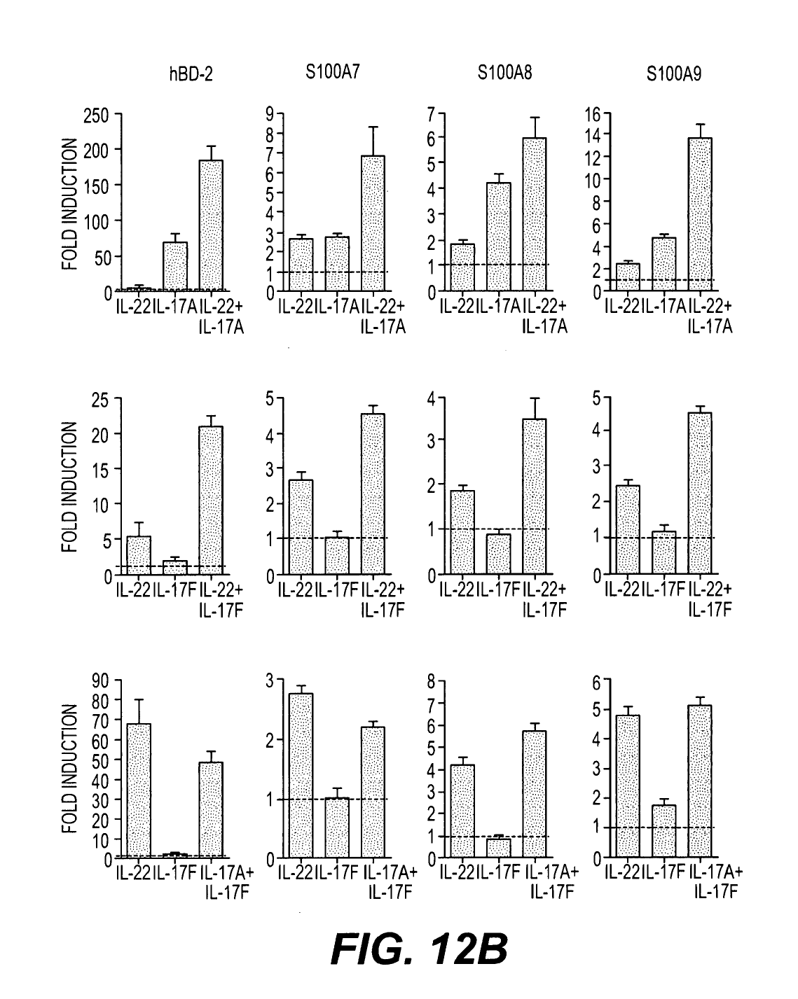

One function of IL-22 is to enhance the expression of anti-microbial peptides associated with host defense, including beta-defensin 2 (hBD-2), S100A7, S100A8, and S100A9 (Wolk et al., Immunity (2004) 21:241-54; Boniface et al., J. Immunol. (2005) 174:3695-3702). To examine whether IL-17A, IL-17F, and IL-22 can act cooperatively to regulate these genes, primary human keratinocytes were treated with IL-22, IL-17A, IL-17F, or with combinations of these cytokines. Specifically, primary human keratinocytes (ScienCell) were cultured in keratinocyte medium (ScienCell) on human fibrinogen coated plates (BD Biosciences). Cells were passaged at 80% confluency and all experiments were done between passages 2-4. For evaluation of cytokine effects, 15,000 cells were seeded into a 24 well plate and allowed to adhere for 48 hrs. Cells were then treated with human IL-22, IL-17A, and IL-17F for 44 hours. RNA was purified and quantitative PCR performed using Taqman Real Time PCR and pre-qualified primer-probes (Applied Biosystems). Relative amounts of hBD-2, S100A7, S100A8, and S100A9 transcript were determined by normalization to GAPDH. Fold induction was calculated relative to expression in keratinocytes that were not treated with any cytokine (denoted by dashed line in FIG. 12). IL-17A induced upregulation of all four anti-microbial peptides examined (5-70 fold induction at 200 ng/ml) (FIG. 12A). IL-22 also induced all four anti-microbial proteins (2-5 fold induction at 200 ng/ml) whereas IL-17F (200 ng/ml) induced hBD-2 by 8 fold, S100A8 by 1.5 fold, and S100A9 by 2 fold but did not upregulate S100A7.

Keratinocytes were then cultured with paired combinations of IL-22, IL-17A, and IL-17F. Human keratinocytes were stimulated with pairwise combinations of IL-22 (200 ng/ml), IL-17A (20 ng/ml), and IL-17F (20 ng/ml) for 44 hours. hBD-2, S100A7, S100A8, and S100A9 mRNA were quantitated as described above. Data are average±SD and are representative of experiments performed on three separate donors. Treatment with IL-22 (200 ng/ml) and IL-17A (20 ng/ml) led to a synergistic increase of hBD-2 (IL-22: 5 fold; IL-17A: 60 fold; IL-22+IL-17A: 180 fold) and S100A9 (IL-22: 2 fold; IL-17A: 5 fold; IL-22+IL-17A: 13 fold) (FIG. 12B). Treatment with IL-22 (200 ng/ml) and IL-17F (20 ng/ml) also synergistically enhanced hBD-2 (IL-22: 5 fold; IL-17F: 2 fold; IL-22+IL-17F: 20 fold). Even though S100A7, S100A8, and S100A9 were not upregulated by IL-17F (20 ng/ml) alone, IL-17F plus IL-22 enhanced the expression of these three peptides by 2 fold over IL-22 alone. These data demonstrate that IL-22 can act cooperatively, either synergistically or additively, with IL-17A or IL-17F. Keratinocytes treated with a combination of IL-17A and IL-17F enhanced S100A8, but did not further enhance expression of hBD-2, S100A7, or S100A9. The combination of IL-17A and IL-17F resulted in less induction of these genes than the combination of IL-22 with IL-17A or IL-17F. Expression of receptors for IL-22 (IL-22R1) or IL-17 (IL-17RA) were not altered with IL-22, IL-17A, or IL-17F treatment, suggesting that these effects are not related to changes in receptor expression. These data demonstrate that IL-22 in combination with IL-17A or IL-17F cooperatively enhances the expression of anti-microbial peptides.

### Example 13

IL-22, IL-17A, IL-17F, and IL-23p19 are upregulated in psoriasis.

The data demonstrate that IL-22 is co-expressed with IL-17A and IL-17F in vivo after immunization with a model antigen. To further examine the relevance of these findings in a human disease, the expression of IL-22, IL-17A, IL-17F, and IL-23p19 were analyzed in psoriasis vulgaris, an inflammatory disease of the skin. Psoriasis is a complex, multigenic disease that affects approximately 2% of the US population and is characterized by the formation of red, raised, scaly lesions (Schon, M. et al., N. Engl J. Med. (2005) 352:1899-1912). While the etiology of psoriasis is still being debated, considerable evidence exists showing that T cells are a pathogenic component of this disease (Christophers, E. et al., Int. Arch. Allergy Immunol. (1996) 110:199-206). T cells are present in lesional skin of psoriasis patients and a variety of T cell derived cytokines have been found to be upregulated in lesional skin (Nickoloff, B. et al., Arch. Dermatol. (1991) 127:871-884). Here, the expression of IL-22, IL-17A, IL-17F, and IL-23p19 was examined in skin from psoriasis patients and the potential correlative expression between these genes was analyzed.

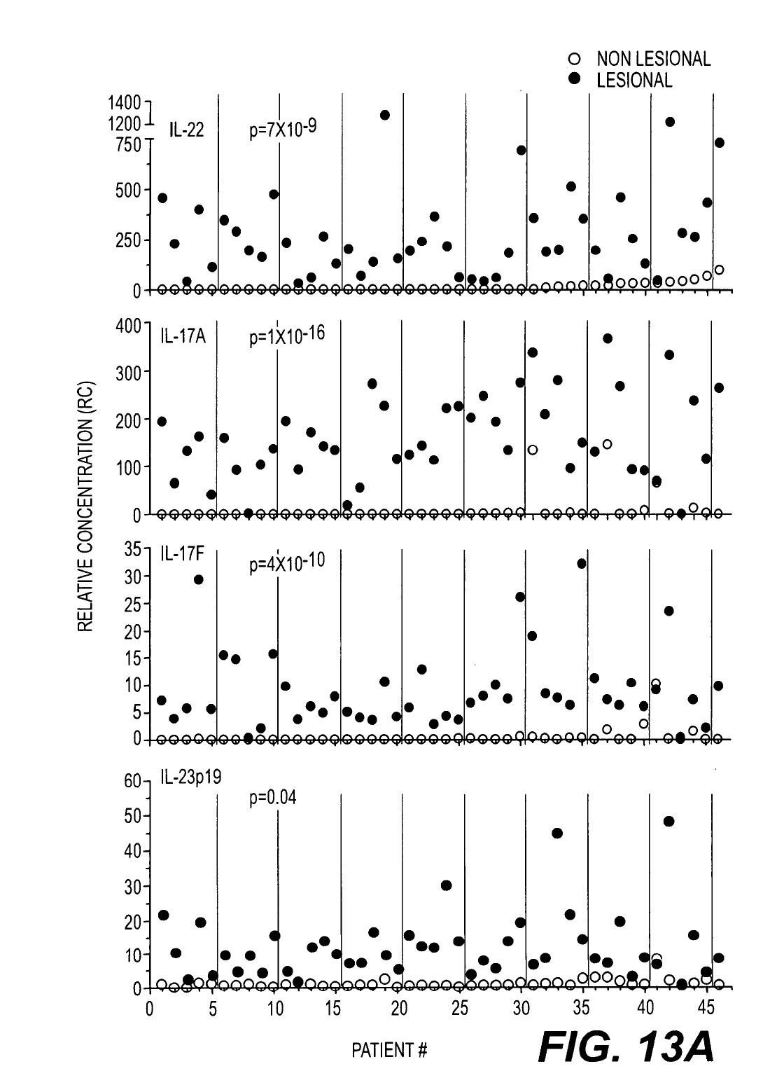

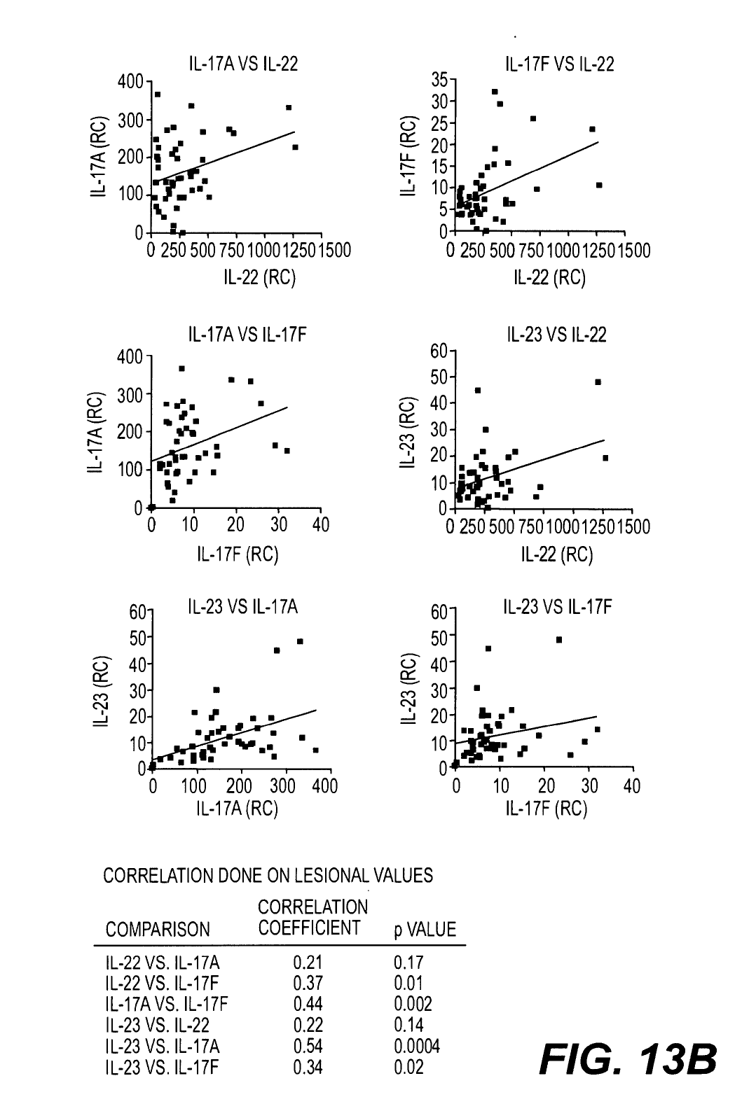

Paired biopsies of non-lesional and lesional skin were obtained from 46 patients with active psoriasis and relative concentrations of IL-22, IL-17A, IL-17F, and IL-23p19 determined by quantitative PCR (FIG. 13A). In non-lesional skin, IL-22 was below the level of detection in 31 of 46 patients. IL-22 was significantly upregulated an average of 25 fold in lesional skin as compared to non-lesional skin (p=7×10⁻⁹), with all 46 patients upregulating IL-22. IL-17A was not detected in 26 of 46 non lesional skin biopsies but was also significantly upregulated 19 fold (p=1×10⁻¹⁶), with 45 out of 46 patients upregulating IL-17A. In 32 of 46 patients, IL-17F was below the level of detection in non-lesional skin. IL-17F was upregulated 21.4 fold (p=4×10⁻¹⁰) with 45 of 46 patients having higher levels of IL-17F in lesional skin. Expression of IL-23p19 was enhanced by 11 (p<0.0001) fold in lesional skin as compared to non-lesional skin, with 44 of 46 patients upregulating IL-23p19. Values were determined by paired Student's t test.

These data are consistent with previous reports demonstrating IL-22 and IL-17A are upregulated in lesional skin of psoriasis patients (Wolk, K. et al., Immunity (2004) 21:241-254; Wolk, K. et al., Eur. J. Immunol. (2006) 36 (5):1309-23; Li, J. et al., J. Huazhong Univ. Sci. Technolog. Med. Sci. (2004) 24:294-296) However, in this study a larger number of patients was analyzed and IL-17A was also examined by quantitative PCR as opposed to the semi-quantitative method used previously. Furthermore, IL-17F, whose expression was previously uncharacterized in psoriasis, is also significantly upregulated in lesional skin. These results suggest that Th17 cytokines play a role in the pathogenesis of psoriasis.

IL-22, IL-17A, IL-17F, and IL-23 were also examined for any correlation in their relative concentrations by performing a Spearman's rank correlation analysis (FIG. 13B). IL-22 exhibited a positive, but not significant, correlation with IL-17A. In contrast, a positive and significant correlation was obtained between IL-22 and IL-17F (0.37, p=0.01) and between IL-17A and IL-17F (0.44, p=0.003). These positive correlation coefficients suggest that there is a correlative relationship between IL-22 and IL-17F and between IL-17A and IL-17F. While the data demonstrate that IL-22 is co-expressed with both IL-17A and IL-17F in vivo in CD4⁺ T cells, expression of these cytokines is not restricted to just lymphocytes. In addition to T cells, IL-17A mRNA has also been detected in neutrophils, eosinophils, and monocytes while IL-22 mRNA is also found in NK cells (Molet, S. et al., J. Allergy Clin. Immunol. (2001) 108:430-438.; Ferretti, S. et al., J. Immunol. (2003) 170:2106-2112.; Awane, M. et al., J. Immunol. (1999) 162:5337-5344.; Wolk, K. et al., Immunity (2004) 21:241-254). Because neutrophils, monocytes, and NK cells have been reported to be present in lesional skin (Schon, M. et al. 2005. N. Engl J. Med. 352:1899-1912), these cells types could also be contributing to the overall IL-22 and IL-17A mRNA in the skin and therefore affect our correlation analysis, especially between IL-22 and IL-17A. However, the positive and significant correlations obtained between IL-22 and IL-17F, as well as between IL-17A and IL-17F, demonstrate a directly proportional relationship between these cytokines in psoriasis. A positive and significant correlation was also detected between IL-23 and IL-17A as well as IL-23 and IL-17F.

### Example 14

### Model for Treatment of Psoriasis

Xenogeneic transplantation in SCID mice is a recognized model for studying psoriasis, see e.g., Boehncke et al., Br. J. Dermatol. (2005) 153 (4):758-66. Under local anesthesia, lesional split-skin (thickness about 0.5 mm) is excised from a patient with chronic plaque-stage psoriasis. Human split grafts are transplanted on the back of 6-8 week old SCID mice. Mice are given 3 weeks to accept the graft and heal. At 22 days following transplantation, mice are injected intraperitoneally with a composition comprising an antagonist of IL-17F alone or an antagonist of IL-22, and at least one IL-17A, IL-17F, or IL-23 antagonist, every other day. As a negative control, mice receive daily intragastric applications of 200 μL PBS and/or isotype control antibody. As a positive control, mice receive daily intragastric application of 2 mg kg⁻¹ dexamethasone in 200 μL PBS. The negative controls develop hallmarks of psoriasis, including acanthosis, papillomatosis, parakeratosis, and a dense mononuclear infiltrate. Mice are sacrificed at day 50 following transplantation and the grafts with surrounding skin are excised. One half of the graft is fixed in formalin and the other half is frozen in liquid nitrogen. Routine hematoxylin and eosin stainings are performed and the pathological changes of the grafts are analyzed both qualitatively (epidermal differentiation) and quantitatively (epidermal thickness, inflammatory infiltrate). The mean epidermal thickness may be measured from the tip of the rete ridges to the border of the viable epidermis using an ocular micrometer. The density of the inflammatory infiltrate may be determined by counting the number of cells in three adjacent power fields. Disease progression may be evaluated using histological analysis to measure hallmarks of psoriasis, such as acanthosis, papillomatosis, parakeratosis, inflammatory infiltrates, and the appearance of the corneal and granular layers.

Negative control mice injected with 200 μL PBS or an isotype-matched control antibody following graft transplantation progressively develop psoriasis. Because psoriatic lesions express higher levels of IL-22, IL-17A, IL-17F, and IL-23p19, treatment with an antagonist of IL-22 and an antagonist of at least one of IL-17A, IL-17F, or IL-23 is expected to suppress or delay psoriasis. Thus, since this model predicts treatment efficacy for psoriasis, treatment with an antagonist of IL-17F alone or an antagonist of IL-22 in combination with an antagonist of at least one of IL-17A, IL-17F, or IL-23 is expected to suppress or delay psoriasis in humans.

### Example 15

### Treatment of Patients

Patients with an autoimmune disorder, respiratory disorder, inflammatory condition of the skin, cardiovascular system, nervous system, kidneys, liver and pancreas or transplant patients may be treated with an antagonist of IL-22 and an antagonist of at least one of IL-17A, IL-17F, or IL-23. Exemplary treatment regimens and expected outcomes are provided below. Dosages and frequency may be adjusted as necessary.

TABLE 1
Treatment Regimens

| | | | | | Expected |
|---|---|---|---|---|---|
| Disorder | Treated with | Dosage | | Frequency | Outcome |
| Multiple | α-IL-22 Ab | 1 | mg/kg | weekly | improvement or |
| Sclerosis | α-IL-17A Ab | | | | stabilization of |
| | | | | | condition |
| Multiple | α-IL-22 Ab | 500 | μg/kg | daily | improvement or |
| Sclerosis | α-IL-17A Ab | | | | stabilization of |
| | | | | | condition |
| Rheumatoid | α-IL-22 Ab | 1 | mg/kg | monthly or | improvement or |
| Arthritis | α-IL-17A Ab | | | bimonthly | stabilization of |
| | | | | | condition |
| Rheumatoid | α-IL-22 Ab | 500 | μg/kg | weekly or | improvement or |
| Arthritis | α-IL-17A Ab | | | biweekly | stabilization of |
| | | | | | condition |
| Asthma | α-IL-22 Ab | 100 | μg/kg | daily | improvement or |
| | α-IL-17A Ab | | | | stabilization of |
| | | | | | condition |
| COPD | α-IL-22 Ab | 100 | μg/kg | daily | improvement or |
| | α-IL-17A Ab | | | | stabilization of |
| | | | | | condition |
| Psoriasis | α-IL-22 Ab | 500 | μg/kg | weekly or | improvement or |
| | α-IL-17A Ab | | | biweekly | stabilization of |
| | | | | | condition |
| Psoriasis | α-IL-22 Ab | 1 | mg/kg | monthly or | improvement or |
| | α-IL-17A Ab | | | bimonthly | stabilization of |
| | | | | | condition |
| Alzheimer's | α-IL-22 Ab | 10 | mg/kg | monthly or | improvement or |
| Disease | α-IL-17A Ab | | | bimonthly | stabilization of |
| | | | | | condition |
| Alzheimer's | α-IL-22 Ab | 1 | mg/kg | weekly or | improvement or |
| Disease | α-IL-17A Ab | | | biweekly | stabilization of |
| | | | | | condition |

In Table 1, the anti-IL-22 antibody can be replaced with a soluble IL-22 receptor or binding protein. The anti-IL-17A antibody in Table 1 can be replaced with an anti-IL-23 antibody, an anti-IL-17F antibody, or a soluble receptor or binding protein for IL-17A, IL-17F, of IL-23.

IL-22 has been characterized as a Th1 cytokine because IL-22 mRNA was found to be upregulated by IL-12 (Wolk et al., J. Immunol. (2002) 168:5397-5402). The work described in this application shows that IL-22 protein is also expressed in the Th17 lineage, revealing a new effector cytokine from Th17 cells. Despite being a Th17 cytokine, IL-22 is located ˜90 kb away from IFN-γ. The distinct expression between IL-22 and IFN-γ suggests cis-regulatory elements exist within this locus that may regulate the differentiation of Th1 versus Th17 cells.

These data also define a new function for IL-23 in inducing IL-22 expression. Although IL-17A is an effector cytokine downstream of IL-23, certain data suggests that IL-17A may not account for all the functions of IL-23. For example, IL-23p19 deficient mice are completely resistant to disease in CIA (Murphy et al., J. Exp. Med. (2003) 198:1951-57). IL-17A deficient mice remain susceptible, albeit with a significantly reduced incidence and severity (Nakae et al., J. Immunol. (2003) 171:6173-77. Also, IL-23p19 deficient mice are susceptible to Citrobacter rodentium infection despite maintaining wild-type expression of IL-17A (Mangan et al., Nature (2006) 441:231-34. These data suggest other cytokines downstream of IL-23 are involved.

IL-22 is upregulated in at least rheumatoid arthritis, psoriasis, and inflammatory bowel disease (Wolk et al., Immunity (2004) 21:241-54; Ikeuchi et al., Arthritis Rheum. (2005) 52:1037-46; Andoh et al., Gastroenterology (2005) 129:969-84). Similar to IL-17A and IL-17F, IL-22 acts directly on epithelial and fibroblast cells in peripheral tissues (Wolk et al., Immunity (2004) 21:241-54; Ikeuchi et al., Arthritis Rheum. (2005) 52:1037-46; Kolls, J. K., and A. Linden. Immunity (2004) 21:467-476. The data demonstrate that IL-22 can function in synergy with IL-17A or IL-17F to enhance the expression of anti-microbial peptides, suggesting that these cytokines cooperate to protect against infection.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

We claim:

1. A method of treating a disorder associated with IL-22, and at least one of IL-17A, IL-17F, or IL-23, in a subject, comprising, administering to the subject a therapeutically effective amount of a composition comprising an antagonist of IL-22, and an antagonist of at least one of IL-17A, IL-17F, or IL-23.

2. The method of claim 1, wherein the antagonist of IL-22 is an antibody or antigen-binding fragment thereof and the antagonist of at least one of IL-17A, IL-17F, or IL-23 is an antibody or antigen-binding fragment thereof.

3. The method of claim 1, wherein the antagonist of IL-22 is a soluble receptor or a binding protein and the antagonist of at least one of. IL-17A, IL-17F, or IL-23 is an antibody or antigen-binding fragment thereof.

4. The method of claim 1, wherein the antagonist of IL-22 is an antibody or antigen-binding fragment thereof and the antagonist of at least one of IL-17A, IL-17F, or IL-23 is a soluble receptor or a binding protein.

5. The method of claim 2, wherein the disorder is chosen from psoriasis, rheumatoid arthritis, juvenile rheumatoid arthritis, osteoarthritis, psoriatic arthritis, ankylosing spondylitis, systemic lupus erythematosis, multiple sclerosis, inflammatory bowel disease, pancreatitis, and Crohn's disease.

6. The method of claim 5, further comprising administering to the subject another therapeutic agent chosen from a cytokine inhibitor, a growth factor inhibitor, an immunosuppressant, an anti-inflammatory agent, a metabolic inhibitor, an enzyme inhibitor, a cytotoxic agent, and a cytostatic agent.

7. The method of claim 6, wherein the therapeutic agent is chosen from a TNF antagonist, an IL-12 antagonist, an IL-15 antagonist, an IL-18 antagonist, an IL-21 antagonist, a T cell depleting agent, a B cell depleting agent, methotrexate, leflunomide, sirolimus (rapamycin) or an analog thereof, a Cox-2 inhibitor, a cPLA2 inhibitor, an NSAID, and a p38 inhibitor.

8. The method of claim 2, wherein the subject is a human.

9. The method of claim 5, wherein the disorder is psoriasis.

10. The method of claim 5, wherein the disorder is psoriasis and wherein the composition comprises an antibody or antigen-binding fragment thereof that binds IL-22 and an antibody or antigen-binding fragment thereof that binds IL-17A or IL-17F.

11. The method of claim 2, wherein the disorder associated with IL-22 is arthritis and wherein the composition comprises an antibody or antigen-binding fragment thereof that binds IL-22 and an antibody or antigen-binding fragment thereof that binds IL-17A or IL-17F.

12. The method of claim 5, wherein the disorder is rheumatoid arthritis and wherein the composition comprises an antibody or antigen-binding fragment thereof that binds IL-22 and an antibody or antigen-binding fragment thereof that binds IL-17A or IL-17F.

13. The method of claim 5, wherein the disorder is inflammatory bowel disease and wherein the composition comprises an antibody or antigen-binding fragment thereof that binds IL-22 and an antibody or antigen-binding fragment thereof that binds IL-17A or IL-17F.

14. The method of claim 5, wherein the disorder is Crohn's disease and wherein the composition comprises an antibody or antigen-binding fragment thereof that binds IL-22 and an antibody or antigen-binding fragment thereof that binds IL-17A or IL-17F.

15. A method of inducing an anti-microbial peptide in a mammalian cell, comprising administering to the mammalian cell IL-22 and IL-17A, IL-22 and IL-17F, or IL-22, IL-17A, and IL-17F in an amount effective to induce an anti-microbial peptide in the mammalian cell.

16. The method of claim 15, wherein the mammalian cell is a keratinocyte.

17. The method of claim 15, wherein the antimicrobial peptide is hBD-2, S100A7, S100A8, or S100A9.

18. The method of claim 16, wherein the antimicrobial peptide is hBD-2, S100A7, S100A8, or S100A9.

19. A method for detecting the presence of IL-22 and at least one of IL-17A, IL-17F, or IL-23 in a sample, in vitro, comprising contacting the sample with a first reagent that binds to IL-22 and a second reagent that binds to IL-17A, IL-17F, or IL-23, and detecting formation of a first complex between the first reagent and the sample and a second complex between the second reagent and the sample, wherein detection of the first complex is indicative of the presence of IL-22 in the sample and detection of the second complex is indicative of the presence of at least one of IL-17A, IL-17F, or IL-23 in the sample.

20. The method of claim 19, wherein the first reagent is a labeled antibody.

21. The method of claim 20, wherein the second reagent is a labeled antibody.

22. The method of claim 21, wherein the sample comprises cells.

23. The method of claim 22, wherein the amount of the first complex detected is proportional to the amount of intracellular IL-22 and the amount of the second complex detected is proportional to the amount of intracellular IL-17A, IL-17F, or IL-23.
